(19) United States
(12) Patent Application Publication
(10) Pub. No.: US 2026/0041683 A1
(43) Pub. Date: Feb. 12, 2026

(54) Compounds with Anti-Acinetobacter Baumannii Activity

(71) Applicant: University of Kentucky Research Foundation, Lexington, KY (US)

(72) Inventor: Sylvie Garneau-Tsodikova, Lexington, KY (US)

(21) Appl. No.: 19/119,272

(22) PCT Filed: Nov. 3, 2023
(86) PCT No.: PCT/US2023/078639

§ 371 Date: Apr. 8, 2025

Related U.S. Application Data
(60) Provisional application No. 63/422,280, filed on Nov. 3, 2022.

(51) Int. Cl. A61K 31/498; A61K 31/166; A61K 31/341; A61K 31/4045; A61K 31/4402; A61K 31/4406; A61K 31/47; A61K 31/4965; A61P 31/14; C07C 243/38; C07D 209/42; C07D 213/56; C07D 215/48; C07D 241/24; C07D 241/44; C07D 307/54

(52) U.S. Cl. CPC A61K 31/498; A61K 31/166; A61K 31/341; A61K 31/4045; A61K 31/4402; A61K 31/4406; A61K 31/47; A61K 31/4965; A61P 31/14; C07C 243/38; C07D 209/42; C07D 213/56; C07D 215/48; C07D 241/24; C07D 241/44; C07D 307/54

(57) ABSTRACT

The presently-disclosed subject matter provides a compound of Formula (I): or a pharmaceutically acceptable salt of the compound, wherein R₁ is C₁₋₄ alkyl, C₁₋₄ alkene, or, wherein R₃, R₄, R₅, and R₆ are independently hydrogen or halogen, or R₄ and R₅, taken together with the atoms to which they are bound, form a 6-membered ring; and wherein X₁ and X₂ are independently C or N; and wherein R₂ is or, wherein R₇ and R₈ are independently hydrogen, halogen, or C₁₋₄ alkoxy; and the use thereof in controlling (Acinetobacter baumannii and treating A. baumannii infection.

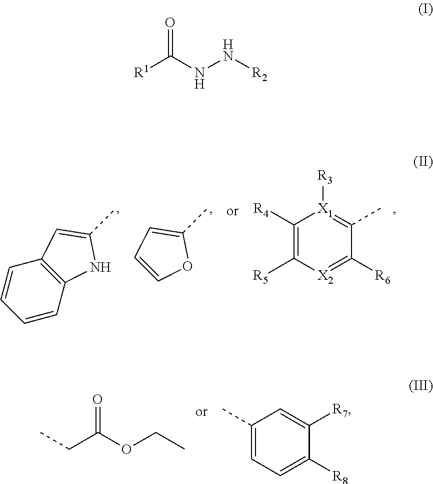

### RELATED APPLICATIONS

This application claims priority from U.S. Provisional Application Ser. No. 63/422,280 filed Nov. 3, 2022, the entire disclosure of which is incorporated herein by this reference.

### TECHNICAL FIELD

The presently-disclosed subject matter relates to antimicrobial compounds, compositions, methods of making, and methods of use thereof. The presently-disclosed subject matter also relates to antimicrobial control of Acinetobacter baumannii. In particular, the presently-disclosed subject matter includes aromatic hydrazides and monohydrides.

### INTRODUCTION

Acinetobacter baumannii (Ab) is an aerobic. Gram-negative bacterium and an extremely concerning nosocomial pathogen.¹ The World Health Organization classified carbapenem-resistant Ab (CRAB) into Priority I (Critical) category for the need of developing new antibiotics.²

A. baumannii belongs to the ESKAPE group of virulent bacteria, which include Enterococcus faecium, Staphylococcus aureus, Klebsiella pneumonia, A. baumannii, Pseudomonas aeruginosa, and Enterobacter spp. The acronym highlights the ability of the bacteria to “escape” antibiotic treatments by developing resistance to many clinically used antibiotics.¹⁶ ESKAPE pathogens have become a major problem in the healthcare system, especially in intensive care units and in immunocompromised patients.^{17. 18}

The number of antibiotics effective against the ESKAPE pathogens has been steadily decreasing.¹⁹ A. baumannii (Ab), particularly, has been noted to cause a variety of infections including the respiratory tract, bacteremia, meningitis, and wound infection, most commonly in war-related trauma.⁴ Ab is extremely effective in colonizing and living on different types of surfaces, which likely accounts for the spread of this bacterium in the hospital setting and outbreaks of its infections.³ Ab is commonly found in intensive care units (ICU), where they are thought to be a cause of ventilator-associated pneumonia, urinary tract infections, surgical site infections, meningitis, and bacteremia.⁶

The mortality rate for Ab infections is 26-56%.⁵ In 2008, Ab, caused 2% of bloodstream infections in relation to the use of catheters, and 8% of ventilator-associated pneumonia, with a mortality rate of 13-30%.²⁵ Based on the Centers for Disease Control (CDC)'s 2019 antibiotic resistance threats report, Ab infections in hospitalized patients, particularly those that are carbapenem-resistant, have not significantly decreased since 2014.²⁶ In 2017, of the 8500 cases, 700 were lethal, and the overall cost of treatment was $281 million USD.

The most significant obstacle in treating Ab infections is its innate and acquired antibiotic resistance, as a result of which multidrug-resistant strains that are highly resistant to most common commercially available antibiotics have emerged.⁷ Indeed, despite extensive antimicrobial treatments available for Ab,²⁰ the development of multidrug resistant Ab has become a worsening problem with the bacteria developing resistance shortly after new drugs are introduced.²¹

Ab uses all types of drug resistance mechanisms: low outer membrane permeability, antibiotic binding-site mutations, antibiotic modification, and efflux.⁴ The acquisition of genes encoding carbapenemases is a highly clinically relevant resistance mechanism for CRAB.^{8, 9} Ab forms biofilms, readily modifies its outer membrane, easily acquires resistance determinants, upregulates efflux pumps, and colonizes medical devices, all of which makes it a difficult bacterial infection to treat.²² The most common resistance mechanism found in Ab is the hydrolysis of β-lactams by β-lactamases.²³

Various antibiotics used to treat Ab include polymyxins, tetracyclines (tigecycline and minocycline), β-lactams in combination with β-lactamase inhibitors (such as sulbactam), and other combinations such as trimethoprim-sulfamethoxazole. However, many Ab strains are highly resistant to these agents. For example, the antibiotic with the highest efficacy is currently polymyxin, however there have been reports of resistance to this antibiotic.²⁴

Furthermore, there are pharmacokinetic (PK) problems with tetracyclines and polymyxins in treating Ab infections, where it is difficult or impossible to achieve high enough drug concentrations in tissues without toxicity.¹⁰ New agents, such as tetracycline eravacycline and cephalosporin cefiderocol, even though now approved for select types of Ab infections, generally appear to not be highly efficacious across different Ab infections.¹⁰⁻¹²

Accordingly, there remains a need in the art for antibiotics with unique modes of action, to avoid cross-resistance with existing drugs, and with a low incidence of resistance emergence to prevent and combat Ab infections.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

A. baumannii (Ab) is one of the highest-priority pathogens due to its ability to mount resistance to numerous antibiotics. Disclosed herein is a series of compounds for use as selective anti-A. baumannii antibiotics.

These monohydrazide compounds represent a unique structural class in antibacterial discovery. They are highly potent against a broad set of A. baumannii strains, including multidrug-resistant isolates, but not against other bacteria. The compounds are not hemolytic, and they inhibit A. baumannii growth in liquid culture and in biofilms. Furthermore, resistance to these compounds did not emerge, even after multiple culture passages. In addition to high anti-bacterial potency, these compounds lack mammalian cytotoxicity.

The presently-disclosed subject matter includes a compound of Formula (I)

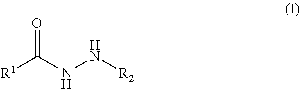

or a pharmaceutically acceptable salt of the compound, wherein R₁ is C₁₋₄ alkyl, C₁₋₄ alkene,

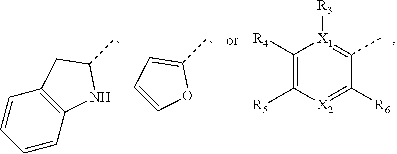

wherein R₃, R₄, R₅, and R₆ are independently hydrogen or halogen, or R₄ and R₅, taken together with the atoms to which they are bound, form a 6-membered ring; and wherein X₁ and X₂ are independently C or N; and wherein R₂ is

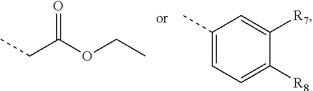

wherein R₇ and R₈ are independently hydrogen, halogen, or C₁₋₄ alkoxy; and the use thereof in controlling Acinetobacter baumannii and treating A. baumannii infection.

The presently-disclosed subject matter includes compounds and compositions comprising one or more of the compounds as disclosed herein. In some embodiments, the pharmaceutical composition includes a compound as disclosed herein and a pharmaceutically-acceptable carrier.

The presently-disclosed subject matter also includes methods of making and using the compounds and compositions as disclosed herein.

The presently-disclosed subject matter also includes a method of controlling an Acinetobacter baumannii, which involves contacting the microbe with an effective amount of a compound or composition as disclosed herein. The presently-disclosed subject matter also includes a method of treating an Acinetobacter baumannii infection, which involves administering to a subject in need thereof an effective amount of a compound or composition as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Synthesis scheme (top) and structures of forty-six (46) exemplary monohydrazide compounds.

FIG. 2. Time-kill curves for Ab ATCC 19606 incubated with compound 3e, a positive control ciprofloxacin (CIP), and in the absence of an inhibitor (labeled Growth control); duplicate assays; CIP (1×MIC, bottom), 3e (1×MIC, middle grey), 3e (4×MIC, middle white), and untreated (top).

FIGS. 3A and 3B. Effect of compound 3e on the biofilm formed by Ab ATCC 19606. FIG. 3A—Compound 3e inhibited biofilm formation. Minimal formation of biofilm at 16 and 32 μg/mL was due to compound aggregation. FIG. 3B—Compound 3e did not disrupt the preformed biofilm.

FIG. 4A-4C. Lack of mammalian cytotoxicity of the monohydrazides in J774A. 1 (FIG. 4A), HEK-293 (FIG. 4B), and Hep G2 (FIG. 4C). Triton™ X-100 (TX) was used as a positive control. Cell growth above 100% was normalized to 100%.

FIG. 5. Lack of resistance of Ab ATCC 19606 to compound 3e (bottom) in a 15-passage MIC assay. Resistance to ciprofloxacin (CIP; top)) readily emerged.

FIG. 6A-D. Mammalian cell cytotoxicity of compounds 1-16a and 1-17e at 32 μg/mL (dark grey bars) and 8 μg/mL (white bars) against HEK-293 (FIG. 6A) and HepG2 (FIG. 6B) cell lines. The corresponding non-normalized plots can be found in FIGS. 6C and 6D).

FIG. 7. Hemolysis of mRBCs treated with 1-16a and 1-17e at 32 μg/mL (dark grey bars) and 8 μg/mL (white bars) for 1 h at 37° C.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds and compositions comprising one or more of the compounds as disclosed herein. The presently-disclosed subject matter also includes methods of making and using the compounds and compositions as disclosed herein. The presently-disclosed subject matter also includes a method of controlling an Acinetobacter baumannii. The presently-disclosed subject matter also includes a method of treating an Acinetobacter baumannii infection.

The presently-disclosed subject matter includes a compound of Formula (I)

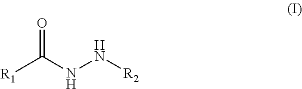

or a pharmaceutically acceptable salt of the compound, wherein R₁ is C₁₋₄ alkyl, C₁₋₄ alkene,

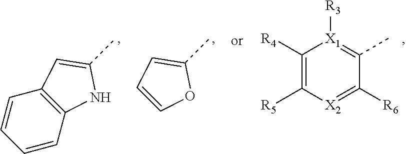

wherein R₃, R₄, R₅, and R₆ are independently hydrogen or halogen, or R₄ and R₅, taken together with the atoms to which they are bound, form a 6-membered ring; and wherein X₁ and X₂ are independently C or N; and wherein R₂ is

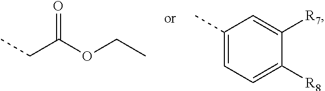

wherein R₇ and R₈ are independently hydrogen, halogen, or C₁₋₄ alkoxy.

In some embodiments, the compound has the structure of Formula (II)

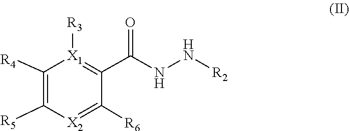

or a pharmaceutically acceptable salt of the compound, wherein R₃, R₄, R₅, and R₆ are independently hydrogen or halogen, or R₄ and R₅, taken together with the atoms to which they are bound, form a 6-membered ring; and wherein X₁ and X₂ are independently C or N; and wherein R₂ is

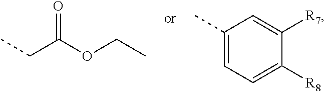

wherein R₇ and R₈ are independently hydrogen, halogen, or C₁₋₄ alkoxy.

In some embodiments, the compound has the structure of Formula (III)

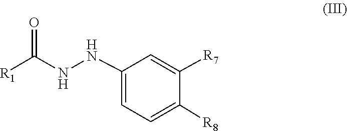

or a pharmaceutically acceptable salt of the compound, wherein R₁ is C₁₋₄ alkyl, C₁₋₄ alkene,

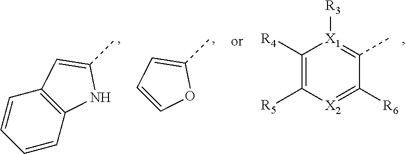

wherein R₃, R₄, R₅, and R₆ are independently hydrogen or halogen, or R₄ and R₅, taken together with the atoms to which they are bound, form a 6-membered ring; and wherein X₁ and X₂ are independently C or N; and R₇ and R₈ are independently hydrogen, halogen, or C₁₋₄ alkoxy.

In some embodiments of the compound of Formula (III), R₁ is

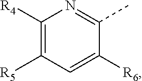

wherein R₄, R₅, and R₆ are independently hydrogen or halogen, or R₄ and R₅, taken together with the atoms to which they are bound, form a 6-membered ring.

In some embodiments of the compound of Formula (III), R₁ is

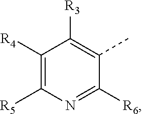

wherein R₃, R₄, R₅, and R₆ are independently hydrogen or halogen, or R₄ and R₅, taken together with the atoms to which they are bound, form a 6-membered ring.

In some embodiments of the compound of Formula (III), R₁ is

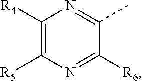

wherein R₄, R₅, and R₆ are independently hydrogen or halogen, or R₄ and R₅, taken together with the atoms to which they are bound, form a 6-membered ring.

In some embodiments of the compound of Formula (III), R₁ is

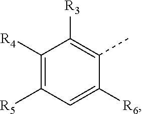

wherein R₃, R₄, R₅, and R₆ are independently hydrogen or halogen, or R₄ and R₅, taken together with the atoms to which they are bound, form a 6-membered ring.

In some embodiments of the compound of Formula (III), R₁ is

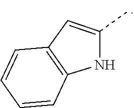

In some embodiments of the compound of Formula (III), R₁ is

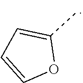

In some embodiments of the compound of Formula (III), R₁ is C₁, C₂, C₃, or C₄ alkyl. In some embodiments of the compound of Formula (III), R₁ is C₁, C₂, C₃, or C₄ alkene.

In some embodiments, the compound has the structure of Formula (IV)

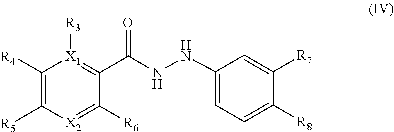

or a pharmaceutically acceptable salt of the compound, wherein R₃, R₄, R₅, and R₆ are independently hydrogen or halogen, or R₄ and R₅, taken together with the atoms to which they are bound, form a 6-membered ring; and wherein X₁ and X₂ are independently C or N; and R₇ and R₈ are independently hydrogen, halogen, or C₁₋₄ alkoxy.

In some embodiments, the compound has the structure of one of the following formulae:

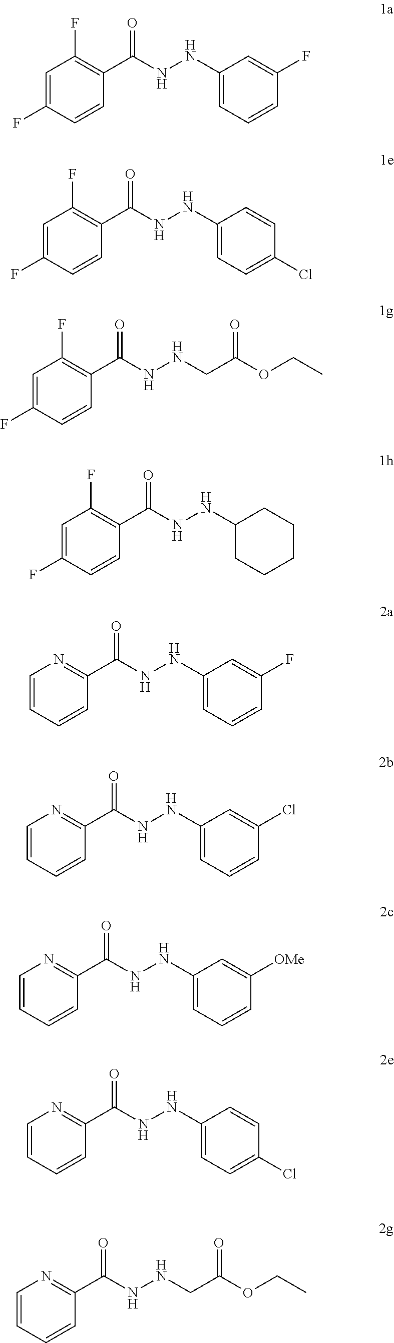

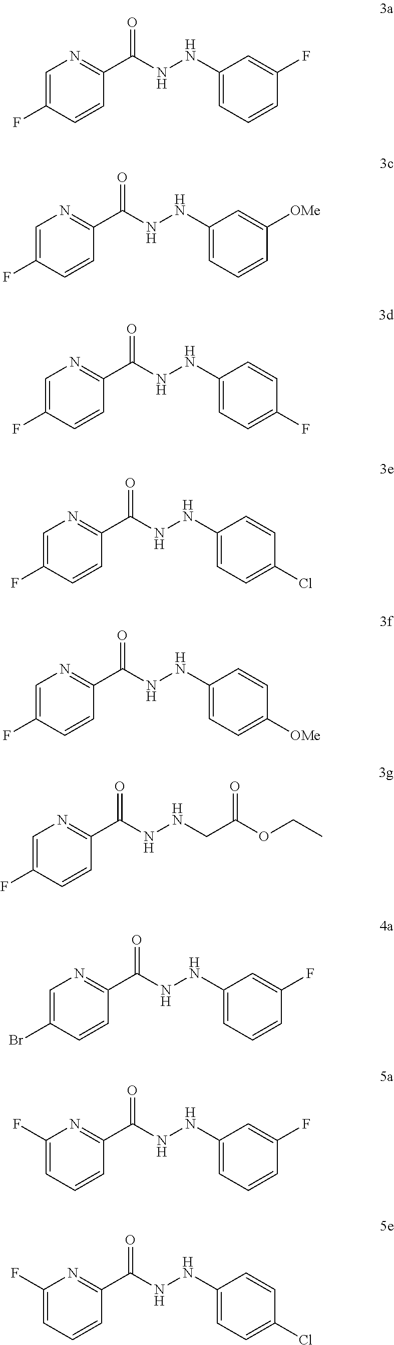

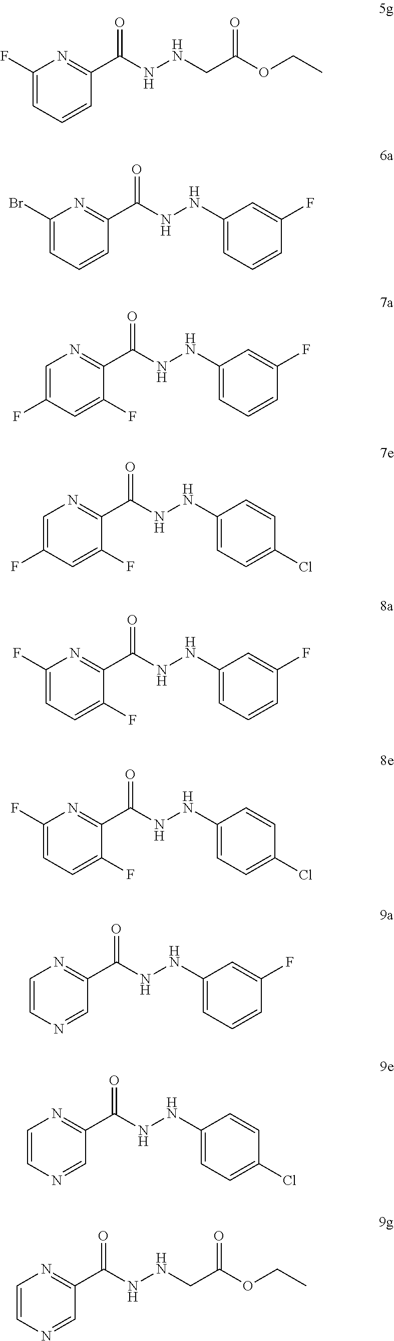

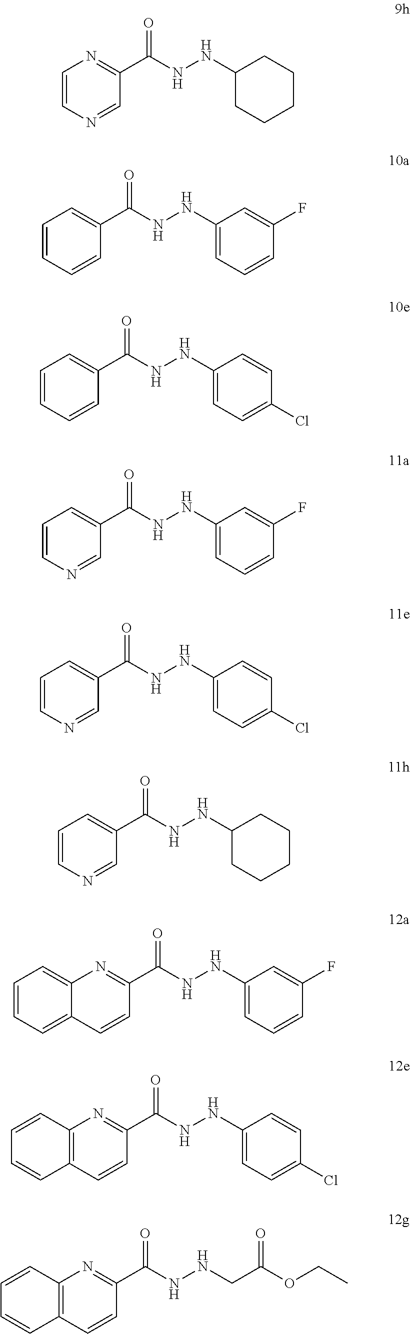

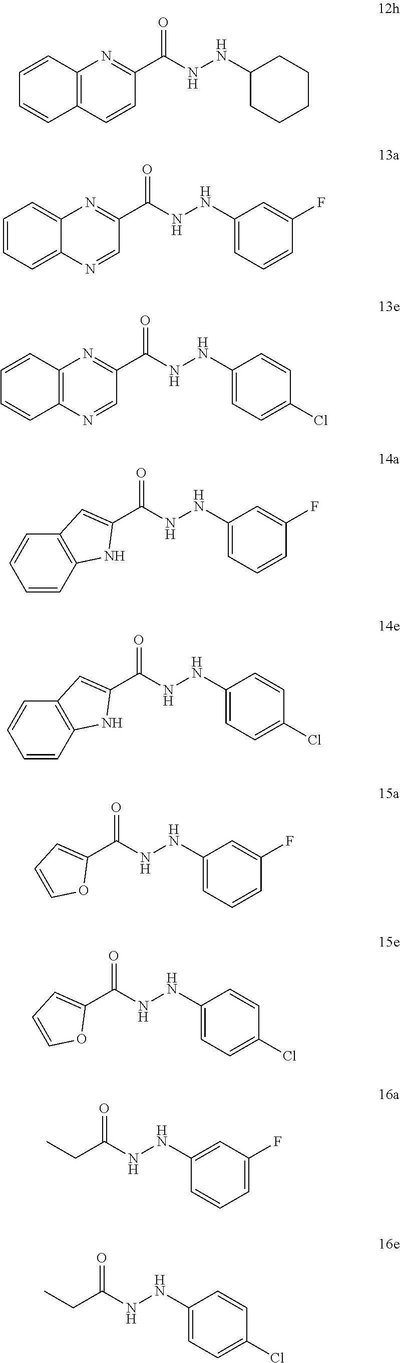

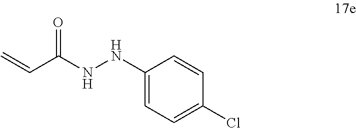

The presently-disclosed subject mailer further includes a pharmaceutical composition comprising the compound of claim 1, and a pharmaceutically-acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, lactose, microcrystalline cellulose, starch, mannitol, glycerin. polyols (such as glycerol, propylene glycol, polyethylene glycol and the like), polymers such as carboxymethylcellulose, hydroxypropyl methylcellulose, or polyvinylpyrrolidone and suitable mixtures thereof, oils such as mineral oil or vegetable oils (such as olive oil), and injectable organic esters such as ethyl oleate.

The presently-disclosed subject matter further includes a method of controlling A. baumannii, which involves contacting the microbe with an effective amount of a compound or composition as disclosed herein. The presently-disclosed subject matter further includes the use of the compounds or compositions as disclosed herein for controlling Acinetobacter baumannii.

The presently-disclosed subject matter further includes a method of treating an A. baumannii infection, which involves administering to a subject in need thereof an effective amount of the compounds or compositions as disclosed herein. The presently-disclosed subject matter further includes the use of the compounds or compositions as disclosed herein for treating an A. baumannii infection. In some embodiments, the compound or composition is administered prophylactically. In some embodiments, the compound or composition is administered therapeutically.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments ±5%, in some embodiments ±1%, in some embodiments±0.5%, in some embodiments±0.1%, in some embodiments±0.010%, and in some embodiments±0.0010% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “alkene” refers to a straight or branched bivalent aliphatic hydrocarbon group having from 1 to 4 carbon atoms, e.g., 1, 2, 3, or 4 carbon atoms. The alkene group can be straight or branched. Exemplary alkene groups include methylene, ethylene, and propylene.

As used herein the term “alkyl” refers to C₁-20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “C₁₋₄ alkyl” refers to an alkyl group having 1 to 4 carbon atoms, i.e., 1, 2, 3, or 4 carbon atoms.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Alkoxyl” refer to an alkyl-O— group wherein alkyl is as previously described. The term “alkoxyl” as used herein can refer to C₁₋₄ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

The terms “halo,” “halide,” or “halogen” as used herein refer to fluoro, chloro, bromo, and iodo groups.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the terms “treatment” or “treating” relate to any treatment of a bacterial infection, including but not limited to prophylactic treatment to prevent development or reduce severity of an infection. The terms “treatment” or “treating” include: (1) preventing an infection from occurring in a subject; (2) inhibiting an infection, i.e., arresting the development or progression of infection; or (3) ameliorating or relieving the symptoms of an infection, i.e., causing regression of one or more of the symptoms.

As will be understood by those of ordinary skill in the art, when the term “prevent” or “prevention” is used in connection with a prophylactic treatment, it should not be understood as an absolute term that would preclude any sign of infection in a subject. Rather, as used in the context of prophylactic treatment, the term “prevent” can refer to inhibiting the development of an infection, limiting the severity of the developed infection, arresting the development of infection, and the like.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. A preparation can be administered therapeutically; that is, administered to treat an existing condition of interest. A preparation can be administered prophylactically; that is, administered for prevention of a condition of interest.

In some embodiments a subject will be administered an effective amount of at least one compound and/or composition provided in the present disclosure. In this respect, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular subject will depend upon a variety of factors including the disorder being treated and the severity of the infection and/or symptoms; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Additionally, the terms “subject” or “subject in need thereof” refer to a target of administration, which optionally displays symptoms related to a particular disease, pathological condition, disorder, or the like. The subject of the herein disclosed methods can be a human, non-human animal. The term does not denote a particular age or sex. Thus, adult and newborn subjects, as well as fetuses, whether male or female, are intended to be covered. The term “subject” includes mammals, and is inclusive of human and veterinary subjects.

The presently-disclosed subject matter is further illustrated by the following specific but non-limiting examples. The following examples may include compilations of data that are representative of data gathered at various times during the course of development and experimentation related to the present invention.

### EXAMPLES

Synthesis and biological testing of a series of representative hydrazide compounds is presented in these Examples. These compounds are selective and highly potent against a broad panel of drug-resistant strains of A. baumannii.

### Example 1: Chemistry Materials and Instrumentation

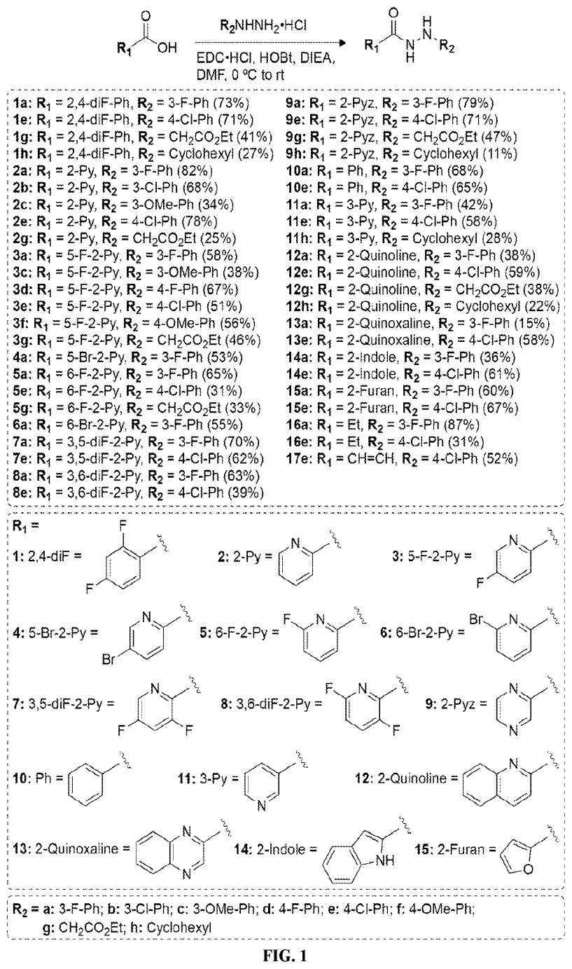

Forty-six exemplary monohydrazide derivatives (FIG. 1) were synthesized for biological testing. These compounds are synthesized in one chemical step by a coupling reaction between carboxylic acids and hydrazines (FIG. 1, synthetic scheme at the top). The reaction yields ranged from 11% to 82%. The compounds included a variety of halogenated and nonhalogenated heterocycles at the R₁ position and a variety of phenyl and other modifications at R₂.

The chemicals used in this study were purchased from Sigma-Aldrich (St. Louis, MO), AK Scientific (Union City, CA), Acros Organics (New Jersey, NJ), TCI America (Portland, OR), Oakwood Chemicals (Estill. SC), Combi-Blocks (San Diego. CA), Accela Chembio (San Diego, CA), and Chem-Impex (Wood Dale, IL), and used without any further purification. Chemical reactions were monitored by TLC (Merck, silica gel 60 F₂₅₄) and visualization was achieved using UV light. Compounds were purified by SiO₂ flash chromatography (Dynamic Adsorbents Inc., flash SiO₂ gel 32-63μ).

¹H and ¹³C NMR spectra were recorded on Agilent VNMRS-500, MR-400, or MR-600 (for both ¹H and ¹³C) spectrometers using deuterated solvents as specified. Chemical shifts (6) are given in parts per million (ppm). Coupling constants (J) are given in Hertz (Hz), and conventional abbreviations used for signal shape are as follows: br s: broad singlet; d, doublet; dd, doublet of doublets; ddd, doublet of doublet of doublets; dt, doublet of triplets; m, multiplet; q, quartet; s, singlet; t, triplet; td, triplet of doublets.

High resolution-mass spectrometry (HRMS) was carried out using a Shimadzu prominence LC system equipped with an AB SCIEX Triple TOF™ 5600 mass spectrometer (Shimadzu manufacturing, Kyoto, Japan). HRMS [M+H]⁺ signals were consistent with the expected molecular weights for all of the reported compounds. Further confirmation of purity for these final molecules was obtained by reversed-phase high-performance liquid chromatography (RP-HPLC) on an Agilent Technologies 1260 Infinity HPLC system by using the following general method: Flow rate=0.5 mL/min; λ=254 nm; column=Vvdac 201SP™ C18, 250×4.6 mm, 90 Å; 5 μm; Eluents: A=H₂O+0.10% TFA, B=MeCN; gradient profile: starting from 5% B, increasing from 5% B to 100% B over 20 min, holding at 100% B for 7 min. decreasing from 100% B to 5% B in 3 min. Prior to each injection, the HPLC column was equilibrated for 15 min with 5% B.

### Example 2: Synthesis of Compound 1a (SGT1786)

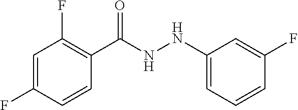 [SMILES: O=C(NNc1cccc(F)c1)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol) and N,N-diisopropylethyl amine (0.33 mL, 1.89 mmol) were added. The reaction mixture was stirred at 0° C. for 30 min followed by the addition of 3-fluorophenylhydrazine hydrochloride (134 mg, 0.82 mmol). The reaction mixture was stirred at room temperature for 12 h, and progress of the reaction was monitored by TLC (2:3/EtOAc:Hexanes. R_{f} 0.41). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO2, 3:7/EtOAc:Hexanes) to afford compound 1a (122 mg, 73%) as a white solid: ¹H NMR (500 MHz, (CD₃)₂SO) δ 10.25 (br s, 1H), 8.32 (br s, 1H), 8.69 (td, J1=8.4 Hz, J2=6.6 Hz, 1H), 7.42 (ddd, J1=10.6 Hz, J2=9.4 Hz, J3=2.5 Hz, 1H), 7.25-7.15 (m, 2H), 6.62 (ddd, J1=8.2 Hz, J2=3.0 Hz, J3=1.5 Hz, 1H), 6.54-6.48 (m, 2H); ¹³C NMR (150 MHz, (CD₃)₂SO) δ 164.5 and 164.4 and 162.8 and 162.7 (dd, J1=248.5 Hz, J2=12.3 Hz), 164.0 and 163.28 (d, J=107.4 Hz), 163.27 and 162.4 (d, J=129.5 Hz), 160.7 and 160.6 and 159.04 and 158.95 (dd, J1=250.2 Hz, J2=12.8 Hz), 151.3 and 151.2 (d, J=10.1 Hz), 131.9 and 131.83 and 131.79 and 131.76 (dd, 11=10.5 Hz, J2=4.4 Hz), 130.5 and 130.4 (d, J=10.3 Hz), 119.44 and 119.41 and 119.34 and 119.31 (dd, J1=15.2 Hz, J2=4.2 Hz), 112.13 and 112.11 and 111.99 and 111.97 (dd, J1=21.6 Hz, J2=3.3 Hz), 108.3, 104.94 and 104.87 and 104.72 and 104.6 (dd, J1=31.6 Hz, J2=10.0 Hz), 104.76, 98.8 and 98.6 (d, J=25.1 Hz); HRMS m/z calcd for C13H9F3N2O [M+H]⁺: 267.2310; found 267.0738; Purity of the compound was further confirmed by HPLC: Rt=15.73 min (98% pure).

### Example 3: Synthesis of Compound 1e (SGT1784)

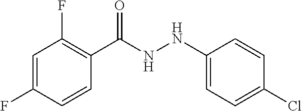 [SMILES: O=C(NNc1ccc(Cl)cc1)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol) and N,N-diisopropylethyl amine (0.33 mL, 1.89 mmol) were added. The reaction mixture was stirred at 0° C. for 30 min followed by the addition of 4-chlorophenylhydrazine hydrochloride (147 mg, 0.82 mmol). The reaction mixture was stirred at room temperature for 12 h, and progress of the reaction was monitored by TLC (2:3/EtOAc:Hexanes. R_{f} 0.39). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:7/EtOAc:Hexanes) to afford compound 1e (126 mg, 71%) as a white solid: ¹H NMR (500 MHz, (CD₃)₂SO) δ 10.24 (d, J=2.6 Hz, 1H), 8.20 (d, J=2.6 Hz, 1H), 7.74 (td, J₁=8.4 Hz, J₂=6.6 Hz, 1H), 7.42 (ddd, J₁=10.6 Hz, J₂=9.4 Hz, J₃=2.5 Hz, 1H), 7.25-7.20 (m, 1H), 7.21 (d, J=8.9 Hz, 2H), 6.80 (d, J=8.9 Hz, 2H): ¹³C NMR (150 MHz, (CD₃)₂SO) δ164.5 and 164.4 and 162.8 and 162.7 (dd, J₁=248.0 Hz, J₂=12.0 Hz), 163.3, 160.7 and 160.6 and 159.05 and 158.96 (dd, J₁=250.3 Hz, J₂=12.9 Hz), 148.0, 131.9 and 131.82 and 131.79 and 131.76 (dd, J₁=9.9 Hz, J₂=4.4 Hz), 128.6 (2C), 122.1, 119.45 and 119.42 and 119.35 and 119.32 (dd, J₁=15.2 Hz, J₂=4.1 Hz), 113.8 (2C), 112.11 and 112.09 and 112.0 and 111.9 (dd, J₁=21.6 Hz, J₂=3.3 Hz), 104.9 and 104.8 and 104.6 (t, J=26.6 Hz); HRMS m/z calcd for C₁₃H₉ClF₂N₂O [M+H]⁺: 283.0449; found 283.0445; Purity of the compound was further confirmed by HPLC: Rₜ=16.25 min (97% pure).

### Example 4: Synthesis of Compound 1g (SGT1811)

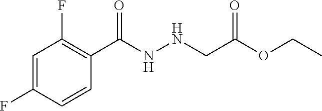 [SMILES: CCOC(=O)CNNC(=O)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg. 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of hydrazinoacetic acid ethyl ester (127 mg, 0.82 mmol). The reaction mixture was stirred at room temperature overnight, and progress of the reaction was monitored by TLC (2:3/EtOAc:Hexanes, R_{f} 0.14). The reaction mixture was quenched with H₂O (100 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 1g (67 mg, 41%) as a white solid: ¹H NMR (500 MHz, (CD₃)₂SO) δ 9.80 (d, J=5.9 Hz, 1H), 7.65 (td, J₁=8.4 Hz, J₂=6.6 Hz, 1H), 7.34 (ddd, J₁=12.0 Hz, J₂=9.4 Hz, J₃=2.5 Hz, 1H), 7.17 (td, J₁=8.4 Hz, J₂=2.0 Hz, 1H), 5.54 (q, J=5.7 Hz, 1H), 4.11 (q, J=7.1 Hz, 2H), 3.62 (d, J=5.3 Hz, 2H), 1.20 (t, J=7.2 Hz, 3H); ¹³C NMR (150 MHz, (CD₃)₂SO) δ 170.3, 164.3 and 164.2 and 162.7 and 162.6 (dd, J₁=248.0 Hz, J₂=12.0 Hz), 161.9, 160.64 and 160.56 and 159.0 and 158.9 (dd, J₁=250.4 Hz, J₂=12.2 Hz), 131.9 and 131.83 and 131.78 and 131.75 (dd, J₁=10.7 Hz, J₂=4.4 Hz), 119.29 and 119.27 and 119.20 and 119.17 (dd, J₁=14.2 Hz, J₂=3.3 Hz), 111.89 and 111.87 and 111.75 and 111.73 (dd, J₁=21.5 Hz, J₂=3.3 Hz), 104.8 and 104.6 and 104.4 (t, J=26.1 Hz), 60.2, 51.9, 14.1; HRMS m % z calcd for C₁₁H₁₂F₂N₂O₃ [M+H]⁺: 259.0894; found 259.0892; Purity of the compound was further confirmed by HPLC: Rₜ=14.73 min (95% pure).

### Example 5: Synthesis of Compound 1h (SGT1887)

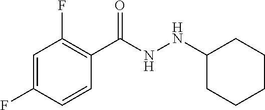 [SMILES: O=C(NNC1CCCCC1)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.35 mL, 1.90 mmol) were added. The reaction mixture was stirred at 0° C. for 30 min followed by the addition of cyclohexylhydrazine hydrochloride (124 mg, 0.82 mmol). The reaction mixture was stirred at room temperature overnight, and progress of the reaction was monitored by TLC (2:3/EtOAc:Hexanes, R_{f} 0.72). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 1h (43 mg, 27%) as a white solid: ¹H NMR (600 MHz, (CD₃)₂SO) δ 9.75 (s, 1H), 7.62 (td, J₁=8.5 Hz, J₂=6.5 Hz, 1H), 7.33 (ddd, J₁=9.3 Hz, J₂=8.2 Hz, J₃=2.5 Hz, 1H), 7.16 (ddd, J₁=10.9 Hz, J₂=8.5 Hz, J₃=2.5 Hz, 1H), 4.97 (s, 1H), 2.79-2.72 (m, 1H). 1.85-1.78 (m, 2H), 1.72-1.67 (m, 2H), 1.57-1.51 (m, 1H), 1.25-1.05 (m, 5H); ₁₃C NMR (150 MHz, (CD₃)₂SO) δ 164.6 and 164.5 and 162.94 and 162.88 (dd, J₁=248.1 Hz, J₂=11.8 Hz), 162.6, 161.0 and 160.9 and 159.3 and 159.2 (dd, J₁=250.5 Hz, J₂=12.7 Hz), 132.15 and 132.12 and 132.08 and 132.05 (dd, J₁=10.2 Hz, J₂=4.7 Hz), 120.41 and 120.38 and 120.31 and 120.28 (dd, J₁=15.3 Hz, J₂=3.9 Hz), 112.29 and 112.27 and 112.2 and 112.1 (dd, J₁=21.6 Hz, J₂=3.3 Hz), 105.2 and 105.0 and 104.8 (t, J=26.2 Hz), 58.3, 31.3 (2C), 26.2, 24.4 (2C); HRMS in z calcd for C₁₃H₁₆F₂N₂O [M+H]⁺: 255.1309; found 255.1310; Purity of the compound was further confirmed by HPLC: Rₜ=14.33 min (95% pure).

### Example 6: Synthesis of Compound 2a (SGT1777)

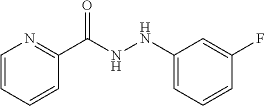 [SMILES: O=C(NNc1cccc(F)c1)c1ccccn1]

To a solution of 2-picolinic acid (100 mg, 0.81 mmol) in DMF (2 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 3-fluorophenylhydrazine hydrochloride (172 mg, 1.06 mmol). The reaction mixture was stirred at room temperature for 12 h, and progress of the reaction was monitored by TLC (2:3/EtOAc:Hexanes, R_{f} 0.41). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2a (153 mg, 82%) as a pale yellow solid: ¹H NMR (500 MHz, (CD₃)₂SO) δ 10.63 (d, J=2.7 Hz, 1H), 8.69 (dt, J₁=4.7 Hz, J₂=1.4 Hz, 1H), 8.21 (d, J=2.7 Hz, 1H), 8.038.01 (m, 2H), 7.65 (td, J₁=4.9 Hz, J₂=3.8 Hz, 1H), 7.15 (td, J₁=8.0 Hz, J₂=6.7 Hz, 1H), 6.58 (ddd, J₁=8.3 Hz, J₂=2.1 Hz, J₃=0.9 Hz, 1H), 6.51-6.43 (m, 2H); ¹³C NMR (150 MHz, (CD₃)₂SO) δ 164.1, 164.0 and 162.4 (d, J=239.2 Hz), 151.54 and 151.47 (d, J=10.8 Hz), 149.6, 148.7, 137.8, 130.34 and 130.27 (d, J=10.3 Hz), 126.9, 122.3, 108.3, 104.6 and 104.5 (d, J=20.7 Hz), 98.8 and 98.6 (d, J=25.1 Hz); HRMS m/z calcd for C₁₂H₁₀FN₃O [M+H]⁺: 232.0886: found 232.0877; Purity of the compound was further confirmed by HPLC: Rₜ=15.25 min (97% pure).

### Example 7: Synthesis of Compound 2b (SGT1787)

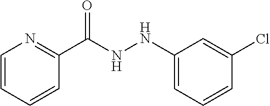 [SMILES: O=C(NNc1cccc(Cl)c1)c1ccccn1]

To a solution of 2-picolinic acid (100 mg, 0.81 mmol) in DMF (3 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 3-chlorophenylhydrazine hydrochloride (190 mg, 1.06 mmol). The reaction mixture was stirred at room temperature for 12 h, and progress of the reaction was monitored by TLC (2:3/EtOAc:Hexanes, R_{f} 0.34). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2b (136 mg, 68%) as a yellow solid: ¹H NMR (500 MHz, (CD₃)₂SO) δ 10.66 (d, J=2.6 Hz, 1H), 8.70 (dt, J₁=4.7 Hz, J₂=1.4 Hz, 1H), 8.22 (d, J=2.5 Hz, 1H), 8.03-8.01 (m, 2H), 7.66 (td, J₁=4.9 Hz, J₂=3.9 Hz, 1H), 7.17-7.13 (m, 2H), 6.74-6.69 (m, 3H); ¹³C NMR (150 MHz, (CD₃)₂SO) a 164.0, 150.9, 149.6, 148.7, 137.8, 133.4, 130.4, 126.9, 122.3, 117.9, 111.5, 110.9; HRMS m/z calcd for C₁₂H₁₀ClN₃O [M+H]+: 248.0590; found 248.0587; Purity of the compound was further confirmed by HPLC: Rₜ=15.83 min (99% pure).

### Example 8: Synthesis of Compound 2c (SGT1772)

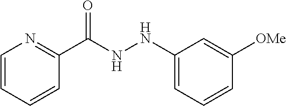 [SMILES: COc1cccc(NNC(=O)c2ccccn2)c1]

To a solution of pyridine-2-carboxylic acid (125 mg, 1.02 mmol) in DMF (3 mL) at 0° C., N-(3-dimethylaminopropyl)-N′-ethylcarbodiimide hydrochloride (234 mg, 1.22 mmol), 1-hydroxybenzotriazole hydrate (165 mg. 1.22 mmol), and N,N-diisopropylethyl amine (0.53 mL, 3.06 mmol) were added. The reaction mixture was stirred at 0° C. for 30 min followed by the addition of 3-methoxyphenylhydrazine hydrochloride (213 mg, 1.22 mmol). The reaction mixture was stirred at room temperature for 12 h, and progress of the reaction was monitored by TLC (2:3/EtOAc:Hexanes, R_{f} 0.24). The reaction mixture was quenched with H₂O (100 mL) and extracted with EtOAc (70 mL). The organic layer was washed with H₂O (70 mL), brine (20 mL), dried over Na₂SO₄, and concentrated under reduced pressure. The residue obtained was purified by flash column chromatography (SiO₂, 3:2/EtOAc:Hexanes) to afford compound 2c (84 mg, 34%) as a pale yellow solid: ¹H NMR (500 MHz, CD₃OD) δ 8.68 (ddd, J₁=4.8 Hz, J₂=1.8 Hz, J₃=1.0 Hz, 1H), 8.10 (dt. J₁=7.9 Hz, J₂=1.1 Hz, 1H), 7.98 (td, J₁=7.7 Hz, J₂=1.7 Hz, 1H), 7.59 (ddd, J₁=7.7 Hz, J₂=4.8 Hz, J₃=1.3 Hz, 1H), 7.08 (td, J₁=8.1 Hz, J₂=0.5 Hz, 1H), 6.47 (ddd, J₁=8.0 Hz, J₂=2.2 Hz, J₃=0.9 Hz, 1H), 6.44 (t, J=2.3 Hz, 1H), 6.40 (ddd, J₁=8.2 Hz, J₂=2.4 Hz, J₃=0.9 Hz, 1H), 3.73 (s, 3H); ¹³C NMR (150 MHz, (CD₃)₂SO) δ 164.0, 160.1, 150.7, 149.8, 148.7, 137.8, 129.5, 126.8, 122.2, 105.1, 103.9, 98.3, 54.8; HRMS m/z calcd for C₁₃H₁₃N₃O₂ [M+H]⁺: 244.1086; found 244.1087; Purity of the compound was further confirmed by HPLC: Rₜ=14.78 min (95% pure).

### Example 9: Synthesis of Compound 2e (SGT1779)

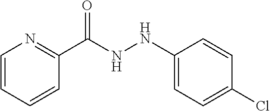 [SMILES: O=C(NNc1ccc(Cl)cc1)c1ccccn1]

To a solution of 2-picolinic acid (100 mg, 0.81 mmol) in DMF (2 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 4-chlorophenylhydrazine hydrochloride (189 mg, 1.06 mmol). The reaction mixture was stirred at room temperature for 12 h, and progress of the reaction was monitored by TLC (2:3/EtOAc:Hexanes, R_{f} 0.39). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2e (156 mg, 78%) as a pale yellow solid: ¹H NMR (500 MHz, (CD₃)₂SO) δ 10.60 (d, J=2.9 Hz, 1H), 8.68-8.66 (m, 1H), 8.08 (d, J=2.8 Hz, 1H), 8.02-7.98 (m, 2H), 7.63 (ddd, J₁=8.7 Hz, J₂=4.8 Hz, J₃=3.5 Hz, 1H), 7.15 (d, J=8.9 Hz, 2H), 6.73 (d, J=8.9 Hz, 2H); ₁₃C NMR (150 MHz, (CD₃)₂SO) δ 164.0, 149.6, 148.7, 148.2, 137.8, 128.5 (2C), 126.9, 122.3, 121.8, 113.8 (2C); HRMS m/z calcd for C₁₂H₁₀ClN₃O [M+H]⁺: 248.0590; found 248.0585; Purity of the compound was further confirmed by HPLC: Rₜ=15.77 min (96% pure).

### Example 10: Synthesis of Compound 2g (SGT2018)

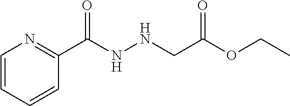 [SMILES: CCOC(=O)CNNC(=O)c1ccccn1]

To a solution of 2-picolinic acid (100 mg, 0.81 mmol) in DMF (3 mL) at 0° C., N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.42 mL, 2.43 mmol) were added. The reaction mixture was stirred at 0° C. for 15 min followed by the addition of hydrazino acetic acid ethyl ester hydrochloride (166 mg, 1.05 mmol). The reaction mixture was stirred at room temperature overnight, and progress of the reaction was monitored by TLC (2:3/EtOAc:Hexanes, R_{f} 0.24). The reaction mixture was quenched with H₂O (100 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes to 4:1/EtOAc:Hexanes) to yield compound 2g (45 mg. 25%) as a gray solid: ¹H NMR (400 MHz, (CD₃)₂SO) δ 10.05 (d, J=6.5 Hz, 1H), 8.62 (m, 1H), 8.00 (d, J=1.4 Hz, 1H), 7.99 (dd, J₁=1.8 Hz, J₂=1.1 Hz, 1H), 7.59 (ddd, J₁=8.0 Hz, J₂=4.8 Hz, J₃=1.7 Hz, 1H), 5.56 (q, J=6.2 Hz, 1H), 4.11 (q, J=7.1 Hz, 2H), 3.65 (d, J=5.8 Hz, 2H), 1.18 (t, J=7.1 Hz, 3H): ³C NMR (100 MHz, (CD₃)₂SO) δ170.6, 161.6, 149.4, 148.5, 137.8, 126.6, 121.8, 60.2, 51.7, 14.0; HRMS m % z calcd for C₁₀H₁₃ClN₃O [M+H]⁺: 224.1035; found 224.1025; Purity of the compound was further confirmed by HPLC: Rₜ=13.85 min (95% pure).

### Example 11: Synthesis of Compound 3a (SGT1440)

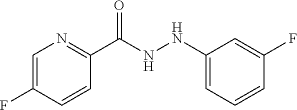 [SMILES: O=C(NNc1cccc(F)c1)c1ccc(F)cn1]

To a solution of 5-fluoro-2-pyridinecarboxylic acid (100 mg, 0.71 mmol) in DMF (3 mL) at 0° C., N-(3-dimethylaminopropyl)-N′-ethylcarbodiimide hydrochloride (163 mg, 0.85 mmol), 1-hydroxybenzotriazole hydrate (115 mg, 0.85 mmol), and N,N-diisopropylethyl amine (0.37 mL, 2.13 mmol) were added. The reaction mixture was stirred at 0° C. for 30 min followed by the addition of 3-fluorophenylhydrazine hydrochloride (138 mg, 0.85 mmol). The reaction mixture was stirred at room temperature for 12 h, and progress of the reaction was monitored by TLC (2:3/EtOAc:Hexanes, R_{f} 0.47). The reaction mixture was quenched with H₂O (100 mL) and extracted with EtOAc (80 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 3a (103 mg, 58%) as a pale yellow solid: ¹H NMR (500 MHz, CD₃OD) δ 8.58 (dt. J₁=2.9 Hz, J₂=0.6 Hz, 1H), 8.18 (ddd, J₁=8.7 Hz, J₂=4.6 Hz, J₃=0.6 Hz, 1H), 7.78 (td, J₁=8.6 Hz, J₂=2.9 Hz, 1H), 7.16 (td, J₁=8.2 Hz, J₂=6.5 Hz, 1H),), 6.66 (ddd, J₁=8.2 Hz, J₂=2.2 Hz, J₃=0.9 Hz, 1H), 6.55 (dt, J₁=11.3 Hz, J₂=2.3 Hz, 1H), 6.52-6.47 (m, 1H); ¹³C NMR (150 MHz, (CD₃)₂SO) δ 163.9 and 162.4 (d, J=138.4 Hz), 163.1, 161.7 and 160.0 (d, J=256.7 Hz), 151.5 and 151.4 (d, J=10.8 Hz), 146.31 and 146.29 (d, J=3.4 Hz), 137.2 and 137.1 (d, J=24.9 Hz), 130.34 and 130.27 (d. J=9.9 Hz), 124.63 and 124.5 (d, J=23.8 Hz), 124.60, 108.3, 104.6 and 104.5 (d, J=20.7 Hz), 98.8 and 98.6 (d, J=25.1 Hz); HRMS in z calcd for C₁₂H₉F₂N₃O [M+H]⁺: 250.0792; found 250.0787; Purity of the compound was further confirmed by HPLC: Rₜ=15.39 min (98% pure).

### Example 12: Synthesis of Compound 3c (SGT1797)

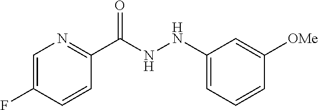 [SMILES: COc1cccc(NNC(=O)c2ccc(F)cn2)c1]

To a solution of 5-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 3-methoxyphenylhydrazine hydrochloride (161 mg, 0.92 mmol). The reaction mixture was stirred at room temperature for 12 h, and progress of the reaction was monitored by TLC (2:3/EtOAc:Hexanes, R_{f} 0.41). The reaction mixture was quenched with H₂O (80 mL) and extracted with EtOAc (60 mL). The organic layer was washed with H₂O (50 mL), brine (20 mL), dried over MgSO₄, and concentrated under reduced pressure. The residue obtained was purified by flash column chromatography (SiO₂, 2:3/EtOAc:Hexanes) to afford compound 3c (70 mg, 38%) as a yellow solid: ¹H NMR (500 MHz, (CD₃)₂SO) δ 10.54 (d, J=3.0 Hz, 1H), 8.69 (d, J=2.9 Hz, 1H), 8.09 (ddd, J₁=8.8 Hz, J₂=4.7 Hz, J₃=0.6 Hz, 1H), 7.92 (td, J₁=8.8 Hz, 0.12=2.9 Hz, 1H), 7.90 (d, J=2.9 Hz, 1H), 7.04 (t, J=8.2 Hz, 1H), 6.35 (ddd, J₁=8.1 Hz, J₂=3.1 Hz, J₃=1.5 Hz, 1H), 6.32-6.27 (m, 2H), 3.66 (s, 3H); ¹³C NMR (100 MHz, (CD₃)₂SO) δ 163.0, 162.0, 160.1, 159.5, 150.6, 146.5 and 146.4 (d. J=3.8 Hz), 137.2 and 136.9 (d, J=24.7 Hz), 129.5, 124.6 and 124.5 and 124.45 and 124.38 (dd, J₁=12.1 Hz, J₂=6.4 Hz), 105.1, 103.9, 98.3, 54.8; HRMS m/z calcd for C₁₃H₁₂FN₃O₂ [M+H]⁺: 262.0992; found 262.0990; Purity of the compound was further confirmed by HPLC: Rₜ=15.23 min (98% pure).

### Example 13: Synthesis of Compound 3d (SGT1799)

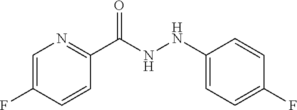 [SMILES: O=C(NNc1ccc(F)cc1)c1ccc(F)cn1]

To a solution of 5-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 4-fluorophenylhydrazine hydrochloride (150 mg, 0.92 mmol). The reaction mixture was stirred at room temperature for 12 h, and progress of the reaction was monitored by TLC (2:3/EtOAc:Hexanes, R_{f} 0.48). The reaction mixture was quenched with H₂O (80 mL) and extracted with EtOAc (60 mL). The organic layer was washed with H₂O (50 mL), brine (20 mL), dried over MgSO₄, and concentrated under reduced pressure. The residue obtained was purified by flash column chromatography (SiO₂, 2:3/EtOAc:Hexanes) to afford compound 3d (118 mg, 67%) as a yellow solid: ¹H NMR (500 MHz, (CD₃)₂SO) δ 10.60 (d, J=3.2 Hz, 1H), 8.69 (d, J=2.9 Hz, 1H), 8.11-8.07 (m, 1H), 7.92 (td, J₁=8.8 Hz, J₂=2.9 Hz, 1H), 7.88 (d, J=3.1 Hz, 1H), 6.98 (t, J=8.9 Hz, 2H), 6.75 (dd, J₁=9.1 Hz, J₂=4.7 Hz, 2H); ¹³C NMR (100 MHz, (CD₃)₂SO) δ 163.1, 162.1 and 159.5 (d, J=257.0 Hz), 157.0 and 154.7 (d, J=232.1 Hz), 146.42 and 146.38 (d, J=4.0 Hz), 145.73 and 145.71 (d, J=1.9 Hz), 137.2 and 136.9 (d, J=24.8 Hz), 124.6 and 124.5 (d, J=4.9 Hz), 124.46 and 124.38 (d. J=7.9 Hz), 115.2 and 115.0 (d, J=22.3 Hz), 113.6 and 113.5 (d, J=7.6 Hz); HRMS m/z calcd C₁₂H₉F₂N₃O [M+H]⁺: 250.0792; found 250.0795; Purity of the compound was further confirmed by HPLC: Rₜ=15.33 min (95% pure).

### Example 14: Synthesis of Compound 3e (SGT1807)

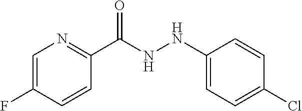 [SMILES: O=C(NNc1ccc(Cl)cc1)c1ccc(F)cn1]

To a solution of 5-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 4-chlorophenylhydrazine hydrochloride (165 mg, 0.92 mmol). The reaction mixture was stirred at room temperature for 12 h, and progress of the reaction was monitored by TLC (2:3/EtOAc:Hexanes, R_{f} 0.48). The reaction mixture was quenched with H₂O (80 mL) and extracted with EtOAc (60 mL). The organic layer was washed with H₂O (50 mL), brine (20 mL), dried over MgSO₄, and concentrated under reduced pressure. The residue obtained was purified by flash column chromatography (SiO₂, 2:3/EtOAc:Hexanes) to afford compound 3e (96 mg, 51%) as a pale yellow solid: ¹H NMR (400 MHz, (CD₃)₂SO) δ 10.62 (d, J=2.8 Hz, 1H), 8.69 (d, J=2.8 Hz, 1H), 8.13-8.06 (m, 2H), 7.92 (td, J₁=8.7 Hz, J₂=2.8 Hz, 1H), 7.17 (d, J=8.8 Hz, 2H), 6.75 (d, J=8.8 Hz, 2H); 13C NMR (150 MHz, (CD₃)₂SO) δ163.1, 161.7 and 160.0 (d, J=256.7 Hz), 148.2, 146.34 and 146.32 (d, J=3.3 Hz), 137.2 and 137.0 (d, J=24.0 Hz), 128.5 (2C), 124.59, 124.55 and 124.5 (d, J=12.9 Hz), 121.8, 113.8 (2C); HRMS m/z calcd for C₁₂H₉ClFN₃O [M+H]⁺: 266.0496; found 266.0493; Purity of the compound was further confirmed by HPLC: Rₜ=15.95 min (95% pure).

### Example 15: Synthesis of Compound 3f (SGT1769)

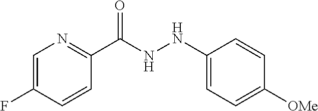 [SMILES: COc1ccc(NNC(=O)c2ccc(F)cn2)cc1]

To a solution of 5-fluoro-2-pyridinecarboxylic acid (100 mg, 0.71 mmol) in DMF (3 mL) at 0° C., N-(3-dimethylaminopropyl)-N′-ethylcarbodiimide hydrochloride (163 mg, 0.85 mmol), 1-hydroxybenzotriazole hydrate (115 mg, 0.85 mmol), and N,N-diisopropylethyl amine (0.37 mL, 2.13 mmol) were added. The reaction mixture was stirred at 0° C. for 30 min followed by the addition of 4-methoxyphenylhydrazine hydrochloride (148 mg, 0.85 mmol). The reaction mixture was stirred at room temperature for 12 h, and progress of the reaction was monitored by TLC (2:3/EtOAc:Hexanes, R_{f} 0.48). The reaction mixture was quenched with H₂O (100 mL) and extracted with EtOAc (80 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 3f (104 mg, 56%) as a pale yellow solid: ¹H NMR (500 MHz, CD₃OD) δ 8.56 (dt, J₁=2.9 Hz, J₂=0.6 Hz, 1H), 8.17 (ddd, J₁=8.8 Hz, J₂=4.6 Hz, J₃=0.6 Hz, 1H), 7.77 (td, J₁=8.6 Hz, J₂=2.9 Hz, 1H), 6.85 (d, J=9.3 Hz, 2H), 6.80 (d, J=9.3 Hz, 2H), 3.72 (s, 3H); ¹³C NMR (150 MHz, (CD₃)₂SO) δ 163.0, 161.6 and 159.9 (d, J=256.7 Hz), 152.8, 146.6 and 146.5 (d, J=4.2 Hz), 143.0, 137.14 and 136.97 (d, J=24.9 Hz), 124.5, 124.43 and 124.39 (d. J=5.3 Hz), 114.2 (2C), 113.9 (2C), 55.3; HRMS m/z calcd for C₁₃H₁₂FN₃O₂ [M+H]⁺: 262.0992; found 262.0989; Purity of the compound was further confirmed by HPLC: Rₜ=14.98 min (98% pure).

### Example 16: Synthesis of Compound 3g (SGT1873)

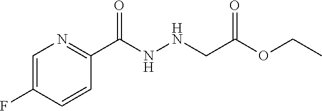 [SMILES: CCOC(=O)CNNC(=O)c1ccc(F)cn1]

To a solution of 5-fluoropicolinic acid (100 mg, 0.71 mmol) in DMF (3 mL) at 0° C., N-(3-dimethylaminopropyl)-N′-ethylcarbodiimide hydrochloride (176 mg. 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 15 min followed by the addition of hydrazino acetic acid ethyl ester (142 mg, 0.92 mmol). The reaction mixture was stirred at room temperature overnight, and progress of the reaction was monitored by TLC (2:3/EtOAc:Hexanes, R_{f} 0.19). The reaction mixture was quenched with H₂O (100 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes to 3:2/EtOAc:Hexanes) to yield compound 3g (79 mg. 46%) as a white solid: ¹H NMR (500 MHz, (CD₃)₂SO) δ 10.04 (d, J=6.5 Hz, 1H), 8.63 (dt, J₁=3.0 Hz, J₂=0.7 Hz, 1H), 8.07 (ddd, J₁=8.7 Hz, 12=4.8 Hz, J=0.7 Hz, 1H), 7.90 (td, J₁=8.7 Hz, J₂=2.9 Hz, 1H), 5.56 (q, J=6.2 Hz, 1H), 4.10 (q, J=7.2 Hz, 2H), 3.65 (d, J=5.9 Hz, 2H), 1.18 (t, J=7.2 Hz, 3H); ¹³C NMR (150 MHz, (CD₃)₂SO) δ 171.1, 162.0 and 160.2 (d, J=256.5 Hz), 161.3, 146.60 and 146.58 (d, J=3.3 Hz), 137.6 and 137.4 (d, J=24.9 Hz), 125.0 and 124.9 (d, J=18.5 Hz), 124.52 and 124.48 (d, J=6.3 Hz), 60.7, 52.1, 14.5; HRMS m/z calcd for C₁₀H₁₂FN₃O₃ [M+H]⁺: 242.0941; found 242.0940; Purity of the compound was further confirmed by HPLC: Rₜ=14.35 min (95% pure).

### Example 17: Synthesis of Compound 4a (SGT1764)

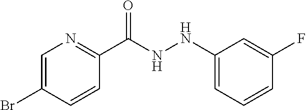 [SMILES: O=C(NNc1cccc(F)c1)c1ccc(Br)cn1]

To a solution of 5-bromo-2-pyridinecarboxylic acid (100 mg, 0.50 mmol) in DMF (3 mL) at 0° C. N-(3-dimethylaminopropyl)-N′-ethylcarbodiimide hydrochloride (113 mg, 0.59 mmol), 1-hydroxybenzotriazole hydrate (80 mg, 0.59 mmol), and N,N-diisopropylethyl amine (0.26 mL, 1.50 mmol) were added. The reaction mixture was stirred at 0° C. for 30 min followed by the addition of 3-fluorophenylhydrazine hydrochloride (96 mg, 0.59 mmol). The reaction mixture was stirred at room temperature for 12 h, and progress of the reaction was monitored by TLC (2:3/EtOAc:Hexanes, R_{f} 0.51). The reaction mixture was quenched with H₂O (100 mL) and extracted with EtOAc (70 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 4a (82 mg, 53%) as a pale yellow solid: ¹H NMR (500 MHz, CD₃OD) δ 8.78 (dd, J₁=2.3 Hz, J₂=0.8 Hz, 1H), 8.20 (dd, J₁=8.4 Hz, J₂=2.3 Hz, 1H), 8.03 (dd, J₁=8.4 Hz, J₂=0.7 Hz, 1H), 7.16 (td, J₁=8.2 Hz, J₂=6.5 Hz, 1H), 6.66 (ddd, J₁=8.2 Hz, J₂=2.2 Hz, J₃=0.9 Hz, 1H), 6.55 (dt, J₁=11.2 Hz, J₂=2.4 Hz, 1H), 6.53-6.48 (m, 1H); ¹³C NMR (150 MHz, (CD₃)₂SO) δ 164.0 and 162.4 (d, J=239.3 Hz), 162.2 and 162.1 (d, J=5.4 Hz), 160.90 and 160.85 and 159.2 and 159.1 (dd, J₁=261.2 Hz, 12=6.4 Hz), 158.4 and 158.3 and 156.6 and 156.5 (dd, J₁=267.6 Hz, J₂=6.6 Hz), 151.3 and 151.2 (d, J=9.8 Hz), 136.49 and 136.46 and 136.41 and 136.39 (dd, J₁=10.7 Hz, J₂=4.3 Hz), 133.89 and 133.86 and 133.74 and 133.71 (dd, J₁=23.6 Hz, J₂=4.9 Hz), 130.5 and 130.4 (d, J=9.8 Hz), 114.2 and 114.0 and 113.9 (t, J=22.1 Hz), 108.3, 104.9 and 104.7 (d, J=20.8 Hz), 98.8 and 98.6 (d, J=25.8 Hz); HRMS m/z calcd for C₁₂H₉BrFN₃O [M+H]⁺: 309.9991; found 309.9989; Purity of the compound was further confirmed by HPLC: Rₜ=16.09 min (97% pure).

### Example 18: Synthesis of Compound 5a (SGT1765)

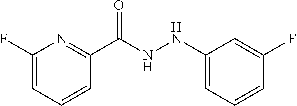 [SMILES: O=C(NNc1cccc(F)c1)c1cccc(F)n1]

To a solution of 6-fluoro-2-pyridinecarboxylic acid (100 mg, 0.71 mmol) in DMF (3 mL) at 0° C., N-(3-dimethylaminopropyl)-N′-ethylcarbodiimide hydrochloride (163 mg, 0.85 mmol), 1-hydroxybenzotriazole hydrate (115 mg, 0.85 mmol), and N,N-diisopropylethyl amine (0.37 mL, 2.13 mmol) were added. The reaction mixture was stirred at 0° C. for 30 min followed by the addition of 3-fluorophenylhydrazine hydrochloride (138 mg, 0.85 mmol). The reaction mixture was stirred at room temperature for 12 h, and progress of the reaction was monitored by TLC (2:3/EtOAc:Hexanes, R_{f} 0.48). The reaction mixture was quenched with H₂O (100 mL) and extracted with EtOAc (70 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 5a (115 mg, 65%) as a pale yellow solid: ¹H NMR (500 MHz, CD₃OD) δ 8.14 (dd, J₁=15.7 Hz, J₂=7.7 Hz, 1H), 8.03 (ddd, J₁=7.5 Hz, J₂=2.3 Hz, J₃=0.9 Hz, 1H), 7.32 (ddd, J₁=8.2 Hz, J₂=2.5 Hz, J₃=0.8 Hz, 1H), 7.16 (td, J₁=8.2 Hz, J₂=6.5 Hz, 1H), 6.66 (ddd, J₁=8.2 Hz, J₂=2.1 Hz, J₃=0.9 Hz, 1H), 6.55 (dt, J₁=11.2 Hz, J₂=2.3 Hz, 1H), 6.53-6.48 (m, 1H): ¹³C NMR (150 MHz, (CD₃)₂SO) δ 164.0 and 162.6 (d, J=207.9 Hz), 162.9, 162.4 and 161.0 (d, J=235.7 Hz), 151.4 and 151.3 (d, J=9.8 Hz), 148.04 and 147.96 (d, J=11.9 Hz), 143.8 and 143.7 (d, J=8.4 Hz), 130.4 and 130.3 (d, J=9.8 Hz), 120.63 and 120.61 (d, J=3.5 Hz), 113.4 and 113.2 (d, J=36.6 Hz), 108.3, 104.7 and 104.6 (d, J=20.8 Hz), 98.9 and 98.7 (d, J=25.2 Hz); HRMS m/z calcd for C₁₂H₉F₂N₃O [M+H]⁺: 250.0792; found 250.0785; Purity of the compound was further confirmed by HPLC: Rₜ=15.40 min (100% pure).

### Example 19: Synthesis of Compound 5e (SGT1808)

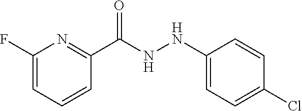 [SMILES: O=C(NNc1ccc(Cl)cc1)c1cccc(F)n1]

To a solution of 6-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 4-chlorophenylhydrazine hydrochloride (165 mg, 0.92 mmol). The reaction mixture was stirred at room temperature for 12 h, and progress of the reaction was monitored by TLC (2:3/EtOAc:Hexanes, R_{f} 0.49). The reaction mixture was quenched with H₂O (80 mL) and extracted with EtOAc (60 mL). The organic layer was washed with H₂O (50 mL), brine (20 mL), dried over MgSO₄, and concentrated under reduced pressure. The residue obtained was purified by flash column chromatography (SiO₂, 2:3/EtOAc:Hexanes) to afford compound 5e (58 mg, 310%) as a pale yellow solid: ¹H NMR (400 MHz, (CD₃)₂SO) δ 10.61 (d, J=2.8 Hz, 1H), 8.20 (td, J₁=8.2 Hz, J₂=7.4 Hz, 1H), 8.10 (d, J=2.8 Hz, 1H), 7.98-7.94 (m, 1H), 7.49-7.45 (m, 1H), 7.18 (d, J=8.9 Hz, 2H), 6.75 (d. J=8.8 Hz, 2H); ¹³C NMR (150 MHz, (CD₃)₂SO) δ 162.8, 162.5 and 160.9 (d, J=238.3 Hz), 148.04, 147.97, 143.8 and 143.7 (d, J=7.7 Hz), 128.5 (2C), 121.9, 120.57 and 120.55 (d, J=3.2 Hz), 113.8 (2C), 113.4 and 113.1 (d, J=36.2 Hz); HRMS m/z calcd for C₁₂H₉ClFN₃O [M+H]⁺: 266.0496; found 266.0489; Purity of the compound was further confirmed by HPLC: Rₜ=15.95 min (97% pure).

### Example 20: Synthesis of Compound 5g (SGT1875)

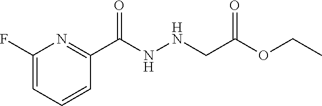 [SMILES: CCOC(=O)CNNC(=O)c1cccc(F)n1]

To a solution of 6-fluoropicolinic acid (100 mg, 0.71 mmol) in DMF (3 mL) at 0° C., N-(3-dimethylaminopropyl)-N′-ethylcarbodiimide hydrochloride (178 mg. 0.93 mmol), 1-hydroxybenzotriazole hydrate (126 mg, 0.93 mmol), and N,N-diisopropylethyl amine (0.37 mL, 2.00 mmol) were added. The reaction mixture was stirred at 0° C. for 15 min followed by the addition of hydrazino acetic acid ethyl ester (110 mg, 0.93 mmol). The reaction mixture was stirred at room temperature overnight, and progress of the reaction was monitored by TLC (2:3/EtOAc:Hexanes, R_{f} 0.15). The reaction mixture was quenched with H₂O (100 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 5g (57 mg, 33%) as a white solid: ¹H NMR (500 MHz, (CD₃)₂SO) δ 10.08 (d, J=6.6 Hz, 1H), 8.18 (td, J₁=8.2 Hz, J₂=7.5 Hz, 1H), 7.93 (ddd, J₁=7.4 Hz, J₂=2.4 Hz, J₃=0.8 Hz, 1H), 7.41 (ddd, J₁=8.3 Hz, J₂=2.5 Hz, J₃=0.8 Hz, 1H), 5.59 (q, J=6.3 Hz, 1H), 4.11 (q, J=7.2 Hz, 2H), 3.65 (d. J=6.0 Hz, 2H), 1.18 (t, J=7.2 Hz, 3H); ¹³C NMR (150 MHz, (CD₃)₂SO) δ 170.5, 162.4 and 160.9 (d, J=238.3 Hz), 160.3, 147.9 and 147.8 (d, J=11.1 Hz), 143.8 and 143.7 (d, J=8.3 Hz), 120.08 and 120.06 (d, J=3.9 Hz), 113.0 and 112.7 (d, J=36.3 Hz), 60.3, 51.5, 14.0; HRMS m/z calcd for C₁₀H₁₂FN₃O₃ [M+H]⁺: 242.0941; found 242.0940; Purity of the compound was further confirmed by HPLC: Rₜ=14.40 min (95% pure).

### Example 21: Synthesis of Compound 6a (SGT1766)

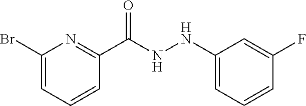 [SMILES: O=C(NNc1cccc(F)c1)c1cccc(Br)n1]

To a solution of 6-bromo-2-pyridinecarboxylic acid (100 mg, 0.50 mmol) in DMF (3 mL) at 0° C., N-(3-dimethylaminopropyl)-N′-ethylcarbodiimide hydrochloride (113 mg, 0.59 mmol), 1-hydroxybenzotriazole hydrate (80 mg, 0.59 mmol), and N,N-diisopropylethyl amine (0.26 mL, 1.50 mmol) were added. The reaction mixture was stirred at 0° C. for 30 min followed by the addition of 3-fluorophenylhydrazine hydrochloride (96 mg, 0.59 mmol). The reaction mixture was stirred at room temperature for 12 h, and progress of the reaction was monitored by TLC (2:3/EtOAc:Hexanes, R_{f} 0.50). The reaction mixture was quenched with H₂O (100 mL) and extracted with EtOAc (70 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 6a (85 mg, 55%) as a pale yellow solid: ¹H NMR (500 MHz, CD₃OD) δ 8.10 (dd, J₁=7.5 Hz, J₂=1.0 Hz, 1H), 7.89 (dd, J₁=8.1 Hz, J₂=7.6 Hz, 1H), 7.82 (dd, J₁=8.0 Hz, J₂=1.0 Hz, 1H), 7.17 (td, J₁=8.3 Hz, J₂=6.5 Hz, 1H), 6.66 (ddd, J₁=8.2 Hz, J₂=2.2 Hz, J₃=0.9 Hz, 1H), 6.55 (dt, J₁=11.2 Hz, J₂=2.3 Hz, 1H), 6.53-6.48 (m, 1H): ¹³C NMR (150 MHz, (CD₃)₂SO) δ 163.9 and 162.3 (d, J=239.2 Hz), 162.9, 151.3 and 151.2 (d, J=10.7 Hz), 150.9, 140.9, 140.4, 131.3, 130.34 and 130.28 (d, J=9.8 Hz), 122.0, 108.3, 104.7 and 104.6 (d, J=20.9 Hz), 98.9 and 98.7 (d. J=25.9 Hz); HRMS m/z calcd for C₁₂H₉BrFN₃O [M+H]⁺: 309.9991; found 309.9981; Purity of the compound was further confirmed by HPLC: Rₜ=15.99 min (100% pure).

### Example 22: Synthesis of Compound 7a (SGT1767)

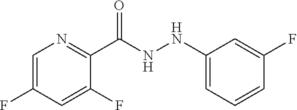 [SMILES: O=C(NNc1cccc(F)c1)c1ncc(F)cc1F]

To a solution of 3,5-difluoro-2-pyridinecarboxylic acid (100 mg, 0.63 mmol) in DMF (3 mL) at 0° C., N-(3-dimethylaminopropyl)-N′-ethylcarbodiimide hydrochloride (144 mg, 0.75 mmol), 1-hydroxybenzotriazole hydrate (102 mg, 0.75 mmol), and N,N-diisopropylethyl amine (0.33 mL, 1.89 mmol) were added. The reaction mixture was stirred at 0° C. for 30 min followed by the addition of 3-fluorophenylhydrazine hydrochloride (122 mg, 0.75 mmol). The reaction mixture was stirred at room temperature for 12 h, and progress of the reaction was monitored by TLC (2:3/EtOAc:Hexanes, R_{f} 0.47). The reaction mixture was quenched with H₂O (100 mL) and extracted with EtOAc (70 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 7a (118 mg, 70%) as a yellow solid: ¹H NMR (500 MHz, CD₃OD) δ 8.50 (dt, J₁=2.4 Hz, J₂=0.6 Hz, 1H), 7.77 (ddd, J₁=11.0 Hz, J₂=8.7 Hz, J₃=2.3 Hz, 1H), 7.17 (td, J₁=8.2 Hz, J₂=6.5 Hz, 1H), 6.69 (ddd, J₁=8.2 Hz, J₂=2.2 Hz, J₃=0.9 Hz, 1H), 6.59 (dt, J₁=11.2 Hz, J₂=2.3 Hz, 1H), 6.53-6.48 (m, 1H); ¹³C NMR (150 MHz, (CD₃)₂SO) δ 164.0 and 162.4 (d, J=239.1 Hz), 163.4, 151.4 and 151.3 (d, J=10.7 Hz), 149.5, 148.4, 140.5, 130.4 and 130.3 (d, J=9.9 Hz), 124.2, 123.8, 108.3, 104.7 and 104.6 (d, J=20.8 Hz), 98.9 and 98.7 (d. J=25.8 Hz); HRMS m/z calcd for C₁₂H₈F₃N₃O [M+H]⁺: 268.0697; found 268.0693; Purity of the compound was further confirmed by HPLC: Rₜ=15.12 min (100% pure).

### Example 23: Synthesis of Compound 7e (SGT1809)

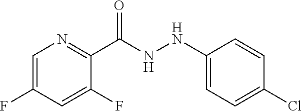 [SMILES: O=C(NNc1ccc(Cl)cc1)c1ncc(F)cc1F]

To a solution of 3,5-difluoro-2-pyridinecarboxylic acid (100 mg, 0.63 mmol) in DMF (3 mL) at 0° C. N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-chlorophenylhydrazine hydrochloride (147 mg, 0.82 mmol). The reaction mixture was stirred at room temperature for 12 h, and progress of the reaction was monitored by TLC (2:3/EtOAc:Hexanes, R_{f} 0.51). The reaction mixture was quenched with H₂O (80 mL) and extracted with EtOAc (60 mL). The organic layer was washed with H₂O (50 mL), brine (20 mL), dried over MgSO₄, and concentrated under reduced pressure. The residue obtained was purified by flash column chromatography (SiO₂, 2:3/EtOAc:Hexanes) to afford compound 7e (110 mg, 62%) as a pale yellow solid: ¹H NMR (400 MHz, (CD₃)₂SO) δ 10.50 (d, J=2.8 Hz, 1H), 8.63 (d, J=2.5 Hz, 1H), 8.19 (d, J=2.8 Hz, 1H), 8.13 (ddd, J₁=10.6 Hz, J₂=9.2 Hz, J₃=2.2 Hz, 1H), 7.21 (d, J=8.8 Hz, 2H), 6.78 (d, J=8.8 Hz, 2H); ¹³C NMR (150 MHz, (CD₃)₂SO) δ 162.11 and 162.07 (d, J=5.4 Hz), 160.9 and 160.8 and 159.11 and 159.07 (dd, J₁=261.2 Hz, J₂=5.6 Hz), 158.34 and 158.29 and 156.54 and 156.50 (dd, J₁=268.5 Hz, J₂=7.5 Hz), 148.0, 136.5 and 136.43 and 136.39 and 136.36 (dd, J₁=10.3 Hz, J₂=4.2 Hz), 133.82 and 133.79 and 133.7 and 133.6 (dd, J₁=23.6 Hz, J₂=5.0 Hz), 128.6 (2C), 122.0, 114.1 and 114.0 and 113.9 (t, J=22.5 Hz), 113.7 (2C); HRMS m/z calcd for C₁₂H₈ClF₂N₃O [M+H]⁺: 284.0402; found 284.0399; Purity of the compound was further confirmed by HPLC: Rₜ=15.63 min (95% pure).

### Example 24: Synthesis of Compound 8a (SGT1768)

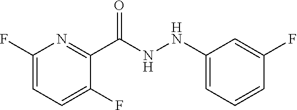 [SMILES: O=C(NNc1cccc(F)c1)c1nc(F)ccc1F]

To a solution of 3,6-difluoro-2-pyridinecarboxylic acid (100 mg, 0.63 mmol) in DMF (3 mL) at 0° C., N-(3-dimethylaminopropyl)-N′-ethylcarbodiimide hydrochloride (144 mg, 0.75 mmol), 1-hydroxybenzotriazole hydrate (102 mg, 0.75 mmol), and N,N-diisopropylethyl amine (0.33 mL, 1.89 mmol) were added. The reaction mixture was stirred at 0° C. for 30 min followed by the addition of 3-fluorophenylhydrazine hydrochloride (122 mg, 0.75 mmol). The reaction mixture was stirred at room temperature for 12 h, and progress of the reaction was monitored by TLC (2:3/EtOAc:Hexanes, R_{f} 0.49). The reaction mixture was quenched with H₂O (100 mL) and extracted with EtOAc (70 mL). The organic layer was washed with H₂O (70 mL), brine (20 mL), dried over Na₂SO₄, and concentrated under reduced pressure. The residue obtained was purified by flash column chromatography (SiO₂, 2:3/EtOAc:Hexanes) to afford compound 8a (105 mg, 63%) as a yellow solid: ¹H NMR (500 MHz, CD₃OD) δ 7.95 (td, J₁=9.1 Hz, J₂=5.9 Hz, 1H), 7.36 (ddd, J₁=9.0 Hz, J₂=3.5 Hz, J₃=2.7 Hz, 1H), 7.18 (td, J₁=8.2 Hz, J₂=6.5 Hz, 1H), 6.68 (ddd, J₁=8.2 Hz, J₂=2.2 Hz, J₃=0.9 Hz, 1H), 6.59 (dt, J₁=11.2 Hz, J₂=2.3 Hz, 1H), 6.54-6.49 (m, 1H); ¹³C NMR (150 MHz, (CD₃)₂SO) δ 164.0 and 162.4 (d, J=239.3 Hz), 161.6 and 161.5 (d, J=4.5 Hz), 157.9 and 156.4 (d, J=236.1 Hz), 156.12 and 156.10 and 154.42 and 154.39 (dd, J₁=256.7 Hz, J₂=4.1 Hz), 151.1 and 151.0 (d, J=10.5 Hz), 136.3 and 136.2 and 136.1 (t, J=14.7 Hz), 132.22 and 132.17 and 132.07 and 132.01 (dd, J₁=22.8 Hz, J₂=8.7 Hz), 130.5 and 130.4 (d, J=9.8 Hz), 114.7 and 114.6 and 114.40 and 114.36 (dd, J₁=41.0 Hz, J₂=6.3 Hz), 108.3, 105.0 and 104.8 (d, J=20.7 Hz), 98.8 and 98.6 (d, J=25.7 Hz); HRMS m/z calcd for C₁₂H₈F₃N₃O [M+H]⁺: 268.0697; found 268.0697; Purity of the compound was further confirmed by HPLC: Rₜ=15.14 min (100% pure).

### Example 25: Synthesis of Compound 8e (SGT1810)

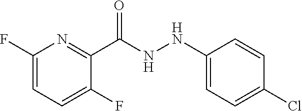 [SMILES: O=C(NNc1ccc(Cl)cc1)c1nc(F)ccc1F]

To a solution of 3,6-difluoro-2-pyridinecarboxylic acid (100 mg, 0.63 mmol) in DMF (3 mL) at 0° C., N-(3-dimethylaminopropyl)-N′-ethylcarbodiimide hydrochloride (157 mg. 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-chlorophenylhydrazine hydrochloride (147 mg, 0.82 mmol). The reaction mixture was stirred at room temperature for 12 h, and progress of the reaction was monitored by TLC (2:3/EtOAc:Hexanes, R_{f} 49). The reaction mixture was quenched with H₂O (80 mL) and extracted with EtOAc (60 mL). The organic layer was washed with H₂O (50 mL), brine (20 mL), dried over MgSO₄, and concentrated under reduced pressure. The residue obtained was purified by flash column chromatography (SiO₂, 2:3/EtOAc:Hexanes) to afford compound 8e (69 mg, 39%) as a pale yellow solid: ¹H NMR (400 MHz, (CD₃)₂SO) δ 10.53 (d, J=2.8 Hz, 1H), 8.21 (d, J=2.8 Hz, 1H), 8.14 (td, J₁=8.9 Hz, 12=6.0 Hz, 1H), 7.52 (dt, J₁=9.0 Hz, J₂=3.1 Hz, 1H), 7.21 (d, J=8.8 Hz, 2H), 6.78 (d, J=8.8 Hz, 2H); ¹³C NMR (150 MHz, (CD₃)₂SO) δ 161.6 and 161.5 (d, J=4.5 Hz), 157.9 and 156.4 (d, J=235.6 Hz), 156.13 and 156.10 and 154.42 and 154.39 (dd, J₁=256.7 Hz, J₂=4.2 Hz), 147.8, 136.3 and 136.2 and 136.1 (t, J=14.8 Hz), 132.23 and 132.17 and 132.08 and 132.02 (dd, J₁=23.0 Hz, J₂=8.9 Hz), 128.6 (2C), 122.2, 114.7 and 114.6 and 114.40 and 114.36 (dd, J₁=41.1 Hz, 12=6.3 Hz), 113.7 (2C); HRMS in z calcd for C₁₂H₈ClF₂N₃O [M+H]⁺: 284.0402; found 284.0399; Purity of the compound was further confirmed by HPLC: Rₜ=15.67 min (96% pure).

### Example 26: Synthesis of Compound 9a (SGT1781)

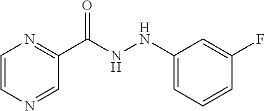 [SMILES: O=C(NNc1cccc(F)c1)c1cnccn1]

To a solution of pyrazinecarboxylic acid (100 mg, 0.81 mmol) in DMF (3 mL) at 0° C. N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.40 mL, 2.43 mmol) were added. The reaction mixture was stirred at 0° C. for 30 min followed by the addition of 3-fluorophenylhydrazine hydrochloride (171 mg, 1.05 mmol). The reaction mixture was stirred at room temperature for 12 h, and progress of the reaction was monitored by TLC (1:2/EtOAc:Hexanes, R_{f} 0.23). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9a (148 mg, 79%) as a yellow solid: ¹H NMR (500 MHz, (CD₃)₂SO) δ 10.81 (s, 1H), 9.18 (d, J=1.6 Hz, 1H), 8.91 (d. J=2.5 Hz, 1H), 8.78 (dd, J₁=2.5 Hz, J₂=1.5 Hz, 1H), 8.29 (s, 1H), 7.19-7.12 (m, 1H), 6.60 (ddd, J₁=8.2 Hz, J₂=3.0 Hz, J₃=1.5 Hz, 1H), 6.53-6.46 (m, 2H); ¹³C NMR (150 MHz, (CD₃)₂SO) δ 164.0, 163.1, 162.4, 151.3 and 151.2 (d, J=10.7 Hz), 147.9, 144.7, 143.7 and 143.6 (d, J=16.2 Hz), 130.4 and 130.3 (d, J=10.4 Hz), 108.4, 104.8 and 104.7 (d. J=21.4 Hz), 98.9 and 98.8 (d, J=25.8 Hz); HRMS m/z calcd for C₁₁H₉FN₄O [M+H]⁺: 233.0838; found 233.0831; Purity of the compound was further confirmed by HPLC: Rₜ=14.50 min (98% pure).

### Example 27: Synthesis of Compound 9e (SGT1783)

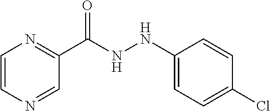 [SMILES: O=C(NNc1ccc(Cl)cc1)c1cnccn1]

To a solution of pyrazinecarboxylic acid (100 mg, 0.81 mmol) in DMF (3 mL) at 0° C. N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.40 mL, 2.43 mmol) were added. The reaction mixture was stirred at 0° C. for 30 min followed by the addition of 4-chlorophenylhydrazine hydrochloride (188 mg, 1.05 mmol). The reaction mixture was stirred at room temperature for 12 h, and progress of the reaction was monitored by TLC (1:2/EtOAc:Hexanes, R_{f} 0.25). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9e (143 mg, 71%) as a brown solid: ¹H NMR (500 MHz, (CD₃)₂SO) δ 10.80 (s, 1H), 9.17 (d, J=1.6 Hz, 1H), 8.91 (d, J=2.6 Hz, 1H), 8.78 (dd, J₁=2.5 Hz, J₂=1.5 Hz, 1H), 8.17 (s, 1H), 7.18 (d, J=8.9 Hz, 2H), 6.77 (d, J=8.9 Hz, 2H): ¹³C NMR (150 MHz, (CD₃)₂SO) δ 163.1, 148.0, 147.8, 144.7, 143.7, 143.6, 128.5 (2C), 122.0, 113.8 (2C); HRMS m/z calcd for C₁₁H₉ClN₄O [M+H]⁺: 249.0543; found 249.0536; Purity of the compound was further confirmed by HPLC: Rₜ=15.04 min (98% pure).

### Example 28: Synthesis of Compound 9g (SGT1812)

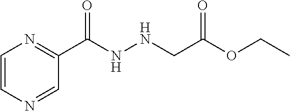 [SMILES: CCOC(=O)CNNC(=O)c1cnccn1]

To a solution of pyrazine-2-carboxylic acid (100 mg, 0.81 mmol) in DMF (3 mL) at 0° C. N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.42 mL, 2.43 mmol) were added. The reaction mixture was stirred at 0° C. for 15 min followed by the addition of hydrazino acetic acid ethyl ester (162 mg, 1.05 mmol). The reaction mixture was stirred at room temperature overnight, and progress of the reaction was monitored by TLC (2:3/EtOAc:Hexanes, R_{f} 0.13). The reaction mixture was quenched with H₂O (100 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes to 4:1/EtOAc:Hexanes) to yield compound 9g (85 mg. 47%) as a white solid: ¹H NMR (500 MHz, (CD₃)₂SO) δ 10.26 (d, J=6.5 Hz, 1H), 9.14 (d, J=1.5 Hz, 1H), 8.86 (d, J=2.5 Hz, 1H), 8.71 (dd, J₁=2.5 Hz, J₂=1.5 Hz, 1H), 5.65 (q, J=6.1 Hz, 1H), 4.10 (q, J=7.1 Hz, 2H), 3.67 (d, J=5.9 Hz, 2H), 1.18 (t, J=7.1 Hz, 3H); ¹³C NMR (150 MHz, (CD₃)₂SO) δ 170.5, 160.6, 147.6, 144.4, 143.5, 143.3, 60.3, 51.6, 14.1; HRMS m/z calcd for C₉H₁₂N₄O₃ [M+H]⁺: 225.0987; found 225.0984; Purity of the compound was further confirmed by HPLC: Rₜ=13.37 min (95% pure).

### Example 29: Synthesis of Compound 9h (SGT1888)

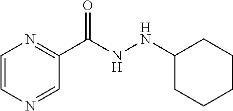 [SMILES: O=C(NNC1CCCCC1)c1cnccn1]

To a solution of pyrazine carboxylic acid (100 mg, 0.81 mmol) in DMF (3 mL) at 0° C., N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.44 mL, 2.42 mmol) were added. The reaction mixture was stirred at 0° C. for 30 min followed by the addition of cyclohexylhydrazine hydrochloride (158 mg, 1.05 mmol). The reaction mixture was stirred at room temperature overnight, and progress of the reaction was monitored by TLC (2:3/EtOAc:Hexanes, R_{f} 0.03). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 1:9/MeOH:CH₂Cl₂) to yield compound 9h (19 mg, 11%) as a light yellow solid: ¹H NMR (600 MHz, (CD₃)₂SO) δ 10.26 (d, J=6.4 Hz, 1H), 9.13 (d, J=1.6 Hz, 1H), 8.85 (d, J=2.4 Hz, 1H), 8.70 (t, J=1.9 Hz, 1H), 5.00 (t, J=6.1 Hz, 1H), 2.82-2.76 (m, 1H), 1.83-1.78 (m, 2H), 1.73-1.66 (m, 2H), 1.58-1.51 (m, 1H), 1.28-1.06 (m, 5H); ¹³C NMR (150 MHz, (CD₃)₂SO) 6162.1, 147.8, 145.3, 143.92, 143.85, 58.4, 31.4 (2C), 26.1, 24.5 (2C); HRMS ml z calcd for C₁₁H₁₆N₄O [M+H]⁺: 221.1402; found 221.1399; Purity of the compound was further confirmed by HPLC: Rₜ=13.41 min (95% pure).

### Example 30: Synthesis of Compound 10a (SGT1881)

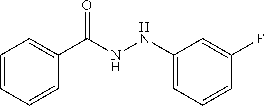 [SMILES: O=C(NNc1cccc(F)c1)c1ccccc1]

To a solution of benzoic acid (100 mg, 0.82 mmol) in DMF (3 mL) at 0° C., N-(3-dimethylaminopropyl)-N′-ethylcarbodiimide hydrochloride (204 mg, 1.06 mmol), 1-hydroxybenzotriazole hydrate (144 mg, 1.06 mmol), and N,N-diisopropylethyl amine (0.45 mL, 2.46 mmol) were added. The reaction mixture was stirred at 0° C. for 30 min followed by the addition of 3-fluorophenylhydrazine hydrochloride (173 mg, 1.06 mmol). The reaction mixture was stirred at room temperature overnight, and progress of the reaction was monitored by TLC (2:3/EtOAc:Hexanes, R_{f} 0.35). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 10a (128 mg, 68%) as a yellow solid: ¹H NMR (600 MHz, (CD₃)₂SO) a 10.04 (s, 1H), 8.22 (s, 1H), 7.92 (d, J=7.0 Hz, 2H), 7.59 (t. J=7.4 Hz, 1H), 7.51 (t, J=7.6 Hz, 2H), 7.16 (dt, J₁=8.7 Hz, J₂=7.8 Hz, 1H), 6.63-6.60 (m, 1H), 6.53-6.47 (m, 2H); ¹³C NMR (150 MHz, (CD₃)₂SO) δ 166.8, 164.5 and 162.9 (d, J=238.4 Hz), 152.3 and 152.2 (d, J=9.9 Hz), 133.3, 132.2, 130.9 and 130.8 (d, J=9.8 Hz), 129.0 (2C), 127.8 (2C), 108.7, 105.2 and 105.1 (d, J=20.8 Hz), 99.3 and 99.1 (d, J=25.1 Hz); HRMS m/z calcd for C₁₃H₁₁FN₂O [M+H]⁺: 231.0933; found 231.0923; Purity of the compound was further confirmed by HPLC: Rₜ=15.42 min (97% pure).

### Example 31: Synthesis of Compound 10e (SGT1813)

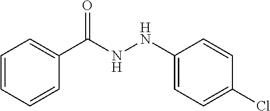 [SMILES: O=C(NNc1ccc(Cl)cc1)c1ccccc1]

To a solution of benzoic acid (100 mg, 0.82 mmol) in DMF (3 mL) at 0° C., N-(3-dimethylaminopropyl)-N′-ethylcarbodiimide hydrochloride (203 mg, 1.06 mmol), 1-hydroxybenzotriazole hydrate (144 mg, 1.06 mmol), and N,N-diisopropylethyl amine (0.43 mL, 2.46 mmol) were added. The reaction mixture was stirred at 0° C. for 30 min followed by the addition of 4-chlorophenylhydrazine hydrochloride (190 mg, 1.06 mmol). The reaction mixture was stirred at room temperature for 12 h, and progress of the reaction was monitored by TLC (2:3/EtOAc:Hexanes, R/0.36). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 10e (131 mg, 65%) as a pale yellow solid: ¹H NMR (500 MHz, (CD₃)₂SO) s 10.40 (d, J=2.5 Hz, 1H), 8.11 (d. J=2.5 Hz, 1H), 7.93-7.89 (m, 2H), 7.58 (tt, J₁=7.3 Hz, J₂=1.3 Hz, 1H), 7.53-7.47 (m, 2H), 7.18 (d, J=8.9 Hz, 2H), 6.78 (d, J=8.9 Hz, 2H); ¹³C NMR (150 MHz, (CD₃)₂SO) δ 166.3, 148.5, 132.8, 131.7, 128.54, 128.51, 127.3, 121.9, 113.8; HRMS m/z calcd for C₁₃H₁₁ClN₂O [M+H]⁺: 247.0638; found 247.0639; Purity of the compound was further confirmed by HPLC: Rₜ=15.88 min (98% pure).

### Example 32: Synthesis of Compound 11a (SGT1882)

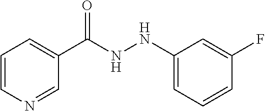 [SMILES: O=C(NNc1cccc(F)c1)c1cccnc1]

To a solution of nicotinic acid (100 mg, 0.81 mmol) in DMF (3 mL) at 0° C., N-(3-dimethylaminopropyl)-N′-ethylcarbodiimide hydrochloride (202 mg, 1.06 mmol), 1-hydroxybenzotriazole hydrate (143 mg, 1.06 mmol), and N,N-diisopropylethyl amine (0.45 mL, 2.44 mmol) were added. The reaction mixture was stirred at 0° C. for 30 min followed by the addition of 3-fluorophenylhydrazine hydrochloride (172 mg, 1.06 mmol). The reaction mixture was stirred at room temperature overnight, and progress of the reaction was monitored by TLC (2:3/EtOAc:Hexanes, R_{f} 0.41). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 0.5:5/MeOH:EtOAc) to yield compound 11a (97 mg, 42%) as a yellow solid: ¹H NMR (600 MHz, (CD₃)₂SO) δ 10.58 (s, 1H), 9.08 (d, J=2.1 Hz, 1H), 8.76 (dd, J₁=4.9 Hz, J₂=1.7 Hz, 1H), 8.29 (s, 1H), 8.26 (dt, J₁=7.9 Hz, J₂=2.0 Hz, 1H), 7.56 (dd, J₁=7.9 Hz, J₂=4.7 Hz, 1H), 7.18 (td, J₁=8.2 Hz, J₂=6.8 Hz, 1H), 6.64 (dd, J₁=8.2 Hz, J₂=2.0 Hz, 1H), 6.55 (dt, J₁=11.6 Hz, J₂=2.3 Hz, 1H), 6.51 (td, J₁=8.5 Hz, J₂=2.5 Hz, 1H); ¹³C NMR (150 MHz, (CD₃)₂SO) a 165.5, 164.5 and 162.9 (d. J=239.1 Hz), 152.9, 152.0 and 151.9 (d, J=9.9 Hz), 148.8, 135.7, 130.94 and 130.87 (d, J=10.3 Hz), 128.9, 124.1, 108.8, 105.4 and 105.3 (d, J=21.7 Hz), 99.4 and 99.2 (d, J=25.3 Hz); HRMS m/z calcd for C₁₂H₁₀FN₃O [M+H]+: 232.0886; found 232.0873; Purity of the compound was further confirmed by HPLC: Rₜ=13.38 min (97% pure).

### Example 33: Synthesis of Compound 11e (SGT1814)

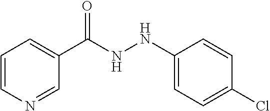 [SMILES: O=C(NNc1ccc(Cl)cc1)c1cccnc1]

To a solution of nicotinic acid (100 mg, 0.81 mmol) in DMF (3 mL) at 0° C., N-(3-dimethylaminopropyl)-N′-ethylcarbodiimide hydrochloride (203 mg, 1.06 mmol), 1-hydroxybenzotriazole hydrate (144 mg, 1.06 mmol), and N,N-diisopropylethyl amine (0.43 mL, 2.46 mmol) were added. The reaction mixture was stirred at 0° C. for 30 min followed by the addition of 4-chlorophenylhydrazine hydrochloride (190 mg, 1.06 mmol). The reaction mixture was stirred at room temperature for 12 h, and progress of the reaction was monitored by TLC (2:3/EtOAc:Hexanes, R_{f} 0.21). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 11e (118 mg, 58%) as a pale yellow solid: ¹H NMR (500 MHz, (CD₃)₂SO) δ 10.58 (br s, 1H), 9.07 (dd, J₁=2.4 Hz, J₂=1.0 Hz, 1H), 8.76 (dd, J₁=4.9 Hz, J₂=1.7 Hz, 1H), 8.25 (ddd, J₁=8.0 Hz, J₂=2.4 Hz, J₃=1.7 Hz, 1H), 8.19-8.17 (m, 1H), 7.55 (ddd, J₁=7.9 Hz, J₂=4.9 Hz, J₃=1.0 Hz, 1H), 7.19 (d, J=8.9 Hz, 2H), 6.81 (d, J=8.9 Hz, 2H); ¹³C NMR (150 MHz, (CD₃)₂SO) s 165.0, 152.4, 148.4, 148.2, 135.2, 128.6, 128.5, 123.7, 122.1, 113.9; HRMS m/z calcd for C₁₂H₁₀ClN₃O [M+H]⁺: 248.0590; found 248.0591; Purity of the compound was further confirmed by HPLC: Rₜ=13.75 min (98% pure).

### Example 34: Synthesis of Compound 11h (SGT1884)

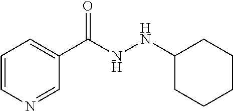 [SMILES: O=C(NNC1CCCCC1)c1cccnc1]

To a solution of nicotinic acid (100 mg, 0.81 mmol) in DMF (3 mL) at 0° C., N-(3-dimethylaminopropyl)-N′-ethylcarbodiimide hydrochloride (202 mg, 1.06 mmol), 1-hydroxybenzotriazole hydrate (143 mg, 1.06 mmol), and N,N-diisopropylethyl amine (0.45 mL, 2.44 mmol) were added. The reaction mixture was stirred at 0° C. for 30 min followed by the addition of cyclohexylhydrazine hydrochloride (159 mg, 1.06 mmol). The reaction mixture was stirred at room temperature overnight, and progress of the reaction was monitored by TLC (2:3/EtOAc:Hexanes, R_{f} 0.07). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 0.5:5/MeOH:EtOAc) to yield compound 11h (50 mg, 28%) as a white solid: ¹H NMR (600 MHz, (CD₃)₂SO) δ 10.14 (d, J=5.6 Hz, 1H), 8.97 (d, J=2.3 Hz, 1H), 8.69 (dd, J₁=4.9 Hz, J₂=1.7 Hz, 1H), 8.16 (dt, J₁=8.0 Hz, J₂=2.0 Hz, 1H), 7.49 (dd, J₁=7.9 Hz, J₂=4.8 Hz, 1H), 5.00 (t, J=5.2 Hz, 1H), 2.80-2.72 (m, 1H), 1.85-1.79 (m, 2H), 1.74-1.67 (m, 2H), 1.57-1.51 (m, 1H), 1.25-1.05 (m. 5H); ¹³C NMR (150 MHz, (CD₃)₂SO) δ 164.5, 152.4, 148.7, 135.3, 129.3, 124.0, 58.5, 31.5 (2C), 26.2, 24.5 (2C); HRMS m/z calcd for C₁₂H₁₇N₃O [M+H]⁺: 220.1450; found 220.1448; Purity of the compound was further confirmed by HPLC: Rₜ=13.03 min (91% pure).

### Example 35: Synthesis of Compound 12a (SGT1876)

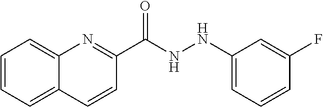 [SMILES: O=C(NNc1cccc(F)c1)c1ccc2ccccc2n1]

To a solution of quinaldic acid (100 mg, 0.58 mmol) in DMF (3 mL) at 0° C., N-(3-dimethylaminopropyl)-N′-ethylcarbodiimide hydrochloride (144 mg. 0.75 mmol), 1-hydroxybenzotriazole hydrate (101 mg, 0.75 mmol), and N,N-diisopropylethyl amine (0.32 mL, 1.74 mmol) were added. The reaction mixture was stirred at 0° C. for 30 min followed by the addition of 3-fluorophenylhydrazine hydrochloride (162 mg, 0.58 mmol). The reaction mixture was stirred at room temperature overnight, and progress of the reaction was monitored by TLC (2:3/EtOAc:Hexanes, R_{f} 0.56). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 12a (63 mg. 38%) as a white solid: ¹H NMR (500 MHz, CD₃OD) δ 8.50 (d, J=8.5 Hz, 1H), 8.24 (d, J=8.6 Hz, 1H), 8.19 (d, J=8.5 Hz, 1H), 8.02 (d, J=8.4 Hz, 1H), 7.86 (ddd, J₁=8.5 Hz, J₂=6.9 Hz, J₃=1.5 Hz, 1H), 7.71 (ddd, J₁=8.2 Hz, J₂=6.9 Hz, J₃=1.3 Hz, 1H), 7.18 (td, J₁=8.2 Hz, J₂=6.4 Hz, 1H), 6.72 (ddd, J₁=8.2 Hz, J₂=2.1 Hz, J₃=0.8 Hz, 1H), 6.62 (dt, J₁=11.3 Hz, J₂=2.4 Hz, 1H), 6.54 (dddd, J₁=11.5 Hz, J₂=8.9 Hz, J₃=2.5 Hz, J₄=0.8 Hz, 1H); ¹³C NMR (150 MHz, (CD₃)₂SO) δ 164.3, 164.0 and 162.4 (d, J=238.4 Hz), 151.5 and 151.4 (d, J=10.7 Hz), 149.8, 146.1, 137.9, 130.6, 130.4 and 130.3 (d, J=10.0 Hz), 129.4, 128.9, 128.3, 128.1, 118.9, 108.3, 104.7 and 104.5 (d, J=21.3 Hz), 98.9 and 98.7 (d, J=25.6 Hz); HRMS m/z calcd for C₁₆H₁₂FN₃O [M+H]⁺: 282.1042; found 282.1039; Purity of the compound was further confirmed by HPLC: Rₜ=16.61 min (100% pure).

### Example 36: Synthesis of Compound 12e (SGT1815)

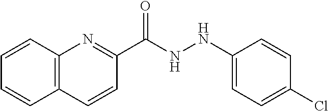 [SMILES: O=C(NNc1ccc(Cl)cc1)c1ccc2ccccc2n1]

To a solution of quinaldic acid (100 mg, 0.58 mmol) in DMF (3 mL) at 0° C., N-(3-dimethylaminopropyl)-N′-ethylcarbodiimide hydrochloride (144 mg, 0.75 mmol), 1-hydroxybenzotriazole hydrate (101 mg, 0.75 mmol), and N,N-diisopropylethyl amine (0.30 mL, 1.74 mmol) were added. The reaction mixture was stirred at 0° C. for 30 min followed by the addition of 4-chlorophenylhydrazine hydrochloride (134 mg, 0.75 mmol). The reaction mixture was stirred at room temperature for 12 h, and progress of the reaction was monitored by TLC (2:3/EtOAc:Hexanes, R/0.47). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 12e (102 mg, 59%) as a pale yellow solid: ¹H NMR (500 MHz, (CD₃)₂SO) δ 10.77 (d, J=2.7 Hz, 1H), 8.59 (dd, J₁=8.6 Hz, J₂=0.8 Hz, 1H), 8.19-8.15 (m, 2H), 8.13-8.09 (m, 1H), 8.11 (d, J=8.5 Hz, 1H), 7.90 (ddd, J₁=8.4 Hz, J₂=6.9 Hz, J₃=1.5 Hz, 1H), 7.75 (ddd, J₁=8.1 Hz, J₂=6.9 Hz, J₃=1.2 Hz, 1H), 7.19 (d, J=8.8 Hz, 2H), 6.81 (d, J=8.8 Hz, 2H); 13C NMR (150 MHz, (CD₃)₂SO) 6164.3, 149.8, 148.2, 146.1, 137.9, 130.6, 129.4, 128.9, 128.5 (2C). 128.3, 128.1, 121.9, 118.9, 113.9 (2C); HRMS m/z calcd for C₁₆H₁₂ClN₃O [M+H]⁺: 298.0747; found 298.0751; Purity of the compound was further confirmed by HPLC: Rₜ=17.11 min (97% pure).

### Example 37: Synthesis of Compound 12g (SGT1816)

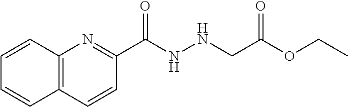 [SMILES: CCOC(=O)CNNC(=O)c1ccc2ccccc2n1]

To a solution of quinaldic acid (100 mg, 0.58 mmol) in DMF (3 mL) at 0° C., N-(3-dimethylaminopropyl)-N′-ethylcarbodiimide hydrochloride (144 mg, 0.75 mmol), 1-hydroxybenzotriazole hydrate (101 mg, 0.75 mmol), and N,N-diisopropylethyl amine (0.30 mL, 1.74 mmol) were added. The reaction mixture was stirred at 0° C. for 30 min followed by the addition of ethyl hydrazinoacetate hydrochloride (116 mg, 0.75 mmol). The reaction mixture was stirred at room temperature for 15 h, and progress of the reaction was monitored by TLC (2:3/EtOAc:Hexanes, R_{f} 0.49). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 12g (60 mg, 38%) as a white solid: ¹H NMR (500 MHz, (CD₃)₂SO) δ 10.20 (d, J=6.4 Hz, 1H), 8.57 (d, J=8.3 Hz, 1H), 8.12 (d. J=8.5 Hz, 1H), 8.12-8.06 (m, 2H), 7.87 (ddd, J₁=8.4 Hz, J₂=6.9 Hz, J₃=1.5 Hz, 1H), 7.72 (ddd, J₁=8.1 Hz, J₂=6.9 Hz, J₃=1.2 Hz, 1H), 5.64 (q, J=6.1 Hz, 1H), 4.13 (q, J=7.2 Hz, 2H), 3.72 (d, J=5.8 Hz, 2H), 1.20 (t, J=7.1 Hz, 3H); ¹³C NMR (150 MHz, (CD₃)₂SO) δ 170.7, 162.0, 149.6, 146.0, 137.9, 130.6, 129.2, 128.8, 128.13, 128.12, 118.6, 60.3, 51.8, 14.1; HRMS m/z calcd for C₁₄H₁₅N₃O₃ [M+H]⁺: 274.1191; found 274.1188; Purity of the compound was further confirmed by HPLC: Rₜ=15.47 min (96% pure).

### Example 38: Synthesis of Compound 12h (SGT1886)

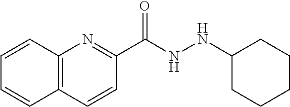 [SMILES: O=C(NNC1CCCCC1)c1ccc2ccccc2n1]

To a solution of quinaldic acid (100 mg, 0.58 mmol) in DMF (3 mL) at 0° C., N-(3-dimethylaminopropyl)-N′-ethylcarbodiimide hydrochloride (144 mg, 0.75 mmol), 1-hydroxybenzotriazole hydrate (101 mg, 0.75 mmol), and N,N-diisopropylethyl amine (0.32 mL, 1.73 mmol) were added. The reaction mixture was stirred at 0° C. for 30 min followed by the addition of cyclohexylhydrazine hydrochloride (113 mg, 0.75 mmol). The reaction mixture was stirred at room temperature overnight, and progress of the reaction was monitored by TLC (2:3/EtOAc:Hexanes, R_{f} 0.48). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 3:10/EtOAc:Hexanes) to yield compound 12h (35 mg, 22%) as a colorless oil: ¹H NMR (600 MHz, (CD₃)₂SO) δ 10.14 (d, J=6.2 Hz, 1H), 8.55 (d, J=8.5 Hz, 1H), 8.13 (d, J=8.5 Hz, 1H), 8.10 (d, J=8.5 Hz, 1H), 8.07 (d, J=8.3 Hz, 1H), 7.86 (t, J=8.5 Hz, 1H), 7.71 (t, J=8.2 Hz, 1H), 4.99 (t, J=6.1 Hz, 1H), 2.89-2.81 (m, 1H), 1.90-1.82 (m, 2H), 1.75-1.68 (m, 2H), 1.58-1.52 (m, 1H), 1.30-1.10 (m, 5H); ¹³C NMR (150 MHz, (CD₃)₂SO) δ 163.4, 150.5, 146.5, 138.2, 130.9, 129.7, 129.2, 128.6, 128.5, 119.2, 58.5, 31.5 (2C). 26.2, 24.5 (2C); HRMS m/z calcd for C₁₆H₁₉N₃O [M+H]+: 270.1606; found 270.1598; Purity of the compound was further confirmed by HPLC: Rₜ=14.94 min (96% pure).

### Example 39: Synthesis of Compound 13a (SGT1879)

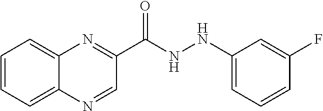 [SMILES: O=C(NNc1cccc(F)c1)c1cnc2ccccc2n1]

To a solution of quinoxaline-2-carboxylic acid (100 mg, 0.58 mmol) in DMF (3 mL) at 0° C., N-(3-dimethylaminopropyl)-N′-ethylcarbodiimide hydrochloride (143 mg, 0.75 mmol), 1-hydroxybenzotriazole hydrate (101 mg, 0.75 mmol), and N,N-diisopropylethyl amine (0.32 mL, 1.74 mmol) were added. The reaction mixture was stirred at 0° C. for 30 min followed by the addition of 3-fluorophenylhydrazine hydrochloride (121 mg, 0.75 mmol). The reaction mixture was stirred at room temperature overnight, and progress of the reaction was monitored by TLC (2:3/EtOAc:Hexanes, R_{f} 0.26). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 13a (24 mg, 15%) as a dark orange solid: ¹H NMR (600 MHz, (CD₃)₂SO) δ 10.95 (s, 1H), 9.45 (s, 1H), 8.35 (s, 1H), 8.26-8.20 (m, 2H), 8.05-7.99 (m, 2H), 7.18 (td, J₁=8.2 Hz, J₂=6.7 Hz, 1H), 6.66 (dd, J₁=8.1 Hz, J₂=2.0 Hz, 1H), 6.57 (dt, J₁=11.7 Hz, J₂=2.2 Hz, 1H), 6.51 (td, J₁=8.3 Hz, 0.2=2.3 Hz, 1H): ¹³C NMR (150 MHz, (CD₃)₂SO) δ 164.5 and 162.9 (d, J=239.3 Hz), 163.9, 151.73 and 151.66 (d, J=9.9 Hz), 144.7, 144.3, 143.5, 140.4, 132.6, 131.8, 130.84 and 130.78 (d, J=9.8 Hz), 130.1, 129.6, 108.9, 105.3 and 105.2 (d, J=20.9 Hz), 99.5 and 99.3 (d, J=25.4 Hz); HRMS m/z calcd for C₁₅H₁₁FN₄O [M+H]⁺: 283.0995; found 283.0999; Purity of the compound was further confirmed by HPLC: Rₜ=15.87 min (97% pure).

### Example 40: Synthesis of Compound 13e (SGT1817)

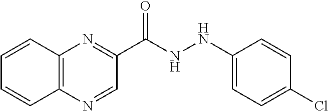 [SMILES: O=C(NNc1ccc(Cl)cc1)c1cnc2ccccc2n1]

To a solution of quinoxaline-2-carboxylic acid (100 mg, 0.58 mmol) in DMF (3 mL) at 0° C., N-(3-dimethylaminopropyl)-N′-ethylcarbodiimide hydrochloride (144 mg, 0.75 mmol), 1-hydroxybenzotriazole hydrate (101 mg, 0.75 mmol), and N,N-diisopropylethyl amine (0.30 mL, 1.71 mmol) were added. The reaction mixture was stirred at 0° C. for 30 min followed by the addition of 4-chlorophenylhydrazine hydrochloride (134 mg. 0.75 mmol). The reaction mixture was stirred at room temperature overnight, and progress of the reaction was monitored by TLC (2:3/EtOAc:Hexanes, Rₜ0.34). The reaction mixture was quenched with H₂O (80 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 13e (101 mg, 58%) as a yellow solid: ¹H NMR (500 MHz, (CD₃)₂SO) δ 10.95 (d, J=2.6 Hz, 1H), 9.44 (s, 1H), 8.25-8.20 (m, 3H), 8.05-7.99 (m, 2H), 7.20 (d, J=8.8 Hz, 2H), 6.84 (d, J=8.8 Hz, 2H); 13C NMR (150 MHz, (CD₃)₂SO) a 163.4, 148.0, 144.3, 143.7, 143.1, 139.9, 132.1, 131.4, 129.6, 129.2, 128.5 (2C), 122.0, 113.9 (2C); HRMS m/z calcd for C₁₅H₁₁ClN₄O [M+H]⁺: 299.0699; found 299.0701; Purity of the compound was further confirmed by HPLC: Rₜ=16.39 min (96% pure).

### Example 41: Synthesis of Compound 14a (SGT1877)

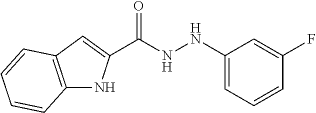 [SMILES: O=C(NNc1cccc(F)c1)c1cc2ccccc2[nH]1]

To a solution of indole-2-carboxylic acid (100 mg, 0.62 mmol) in DMF (3 mL) at 0° C., N-(3-dimethylaminopropyl)-N′-ethylcarbodiimide hydrochloride (155 mg, 0.81 mmol), 1-hydroxybenzotriazole hydrate (109 mg, 0.81 mmol), and N,N-diisopropylethyl amine (0.34 mL, 1.86 mmol) were added. The reaction mixture was stirred at 0° C. for 30 min followed by the addition of 3-fluorophenylhydrazine hydrochloride (102 mg, 0.63 mmol). The reaction mixture was stirred at room temperature overnight, and progress of the reaction was monitored by TLC (2:3/EtOAc:Hexanes, R_{f} 0.58). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 14a (60 mg, 36%) as a light orange solid: ¹H NMR (500 MHz, CD₃OD) δ 7.64 (d. J=8.05 Hz, 1H), 7.46 (dd, J₁=8.3 Hz, J₂ 1.0 Hz, 1H), 7.24 (ddd, J₁=8.4 Hz, J₂=7.0 Hz, J₃=1.1 Hz, 1H), 7.19 (d, J=1.0 Hz, 1H), 7.17 (td, J₁=8.3 Hz, J₂=6.5 Hz, 1H), 7.09 (ddd, J₁=8.1 Hz, J₂=7.0 Hz, J₃=1.0 Hz, 1H), 6.68 (ddd, J₁=8.3 Hz, J₂=7.4 Hz, J₃=0.9 Hz, 1H), 6.57 (dt. J₁=11.3 Hz, J₂=2.3 Hz, 1H), 6.50 (dddd, J₁=11.4 Hz, J₂=8.9 Hz, J₃=2.5 Hz, J₄=0.9 Hz, 1H); ¹³C NMR (150 MHz, (CD₃)₂SO) δ 164.0 and 162.4 (d, J=238.4 Hz), 161.4, 151.8 and 151.7 (d, J=10.5 Hz), 136.6, 130.5 and 130.4 (d, J=9.9 Hz), 129.6, 127.0, 123.6, 121.6, 119.9, 112.3, 108.1, 104.7 and 104.5 (d, J=21.4 Hz), 103.1, 98.7 and 98.5 (d. J=25.0 Hz); HRMS m/z calcd for C₁₅H₁₂FN₃O [M+H]⁺: 270.1042; found 270.1047; Purity of the compound was further confirmed by HPLC: Rₜ=15.86 min (95% pure).

### Example 42: Synthesis of Compound 14e (SGT1818)

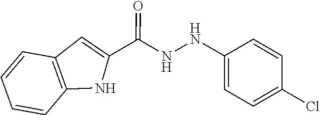 [SMILES: O=C(NNc1ccc(Cl)cc1)c1cc2ccccc2[nH]1]

To a solution of indole-2-carboxylic acid (100 mg, 0.62 mmol) in DMF (3 mL) at 0° C., N-(3-dimethylaminopropyl)-N′-ethylcarbodiimide hydrochloride (155 mg, 0.81 mmol), 1-hydroxybenzotriazole hydrate (109 mg, 0.81 mmol), and N,N-diisopropylethyl amine (0.32 mL, 1.86 mmol) were added. The reaction mixture was stirred at 0° C. for 30 min followed by the addition of 4-chlorophenylhydrazine hydrochloride (145 mg, 0.81 mmol). The reaction mixture was stirred at room temperature overnight, and progress of the reaction was monitored by TLC (2:3/EtOAc:Hexanes, R/0.26). The reaction mixture was quenched with H₂O (80 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 14e (108 mg, 61%) as a yellow solid: ¹H NMR (500 MHz, (CD₃)₂SO) δ 11.67 (s, 1H), 10.40 (d, J=2.1 Hz, 1H), 8.17 (d, J=2.0 Hz, 1H), 7.64 (d, J=8.0 Hz, 1H), 7.44 (dd, J₁=8.3 Hz, J₂ 1.0 Hz, 1H), 7.28-7.25 (m, 1H), 7.23-7.17 (m, 3H), 7.06 (ddd, J₁=8.1 Hz, J₂=7.0 Hz, J₃=1.1 Hz, 1H), 6.79 (d, J=8.9 Hz, 2H); ¹³C NMR (150 MHz, (CD₃)₂SO) δ 161.4, 148.5, 136.6, 129.7, 128.6, 127.0, 123.6, 121.8 (2C), 121.6, 119.9, 113.7 (2C), 112.4, 103.1; HRMS m/z calcd for C₁₅H₁₂ClN₃O [M+H]⁺: 286.0747; found 286.0747; Purity of the compound was further confirmed by HPLC: Rₜ=16.29 min (98% pure).

### Example 43: Synthesis of Compound 15a (SGT1880)

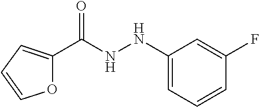 [SMILES: O=C(NNc1cccc(F)c1)c1ccco1]

To a solution of 2-furoic acid (100 mg, 0.89 mmol) in DMF (3 mL) at 0° C., N-(3-dimethylaminopropyl)-N′-ethylcarbodiimide hydrochloride (222 mg, 1.16 mmol), 1-hydroxybenzotriazole hydrate (157 mg, 1.16 mmol), and N,N-diisopropylethyl amine (0.49 mL, 2.68 mmol) were added. The reaction mixture was stirred at 0° C. for 30 min followed by the addition of 3-fluorophenylhydrazine hydrochloride (186 mg, 1.16 mmol). The reaction mixture was stirred at room temperature overnight, and progress of the reaction was monitored by TLC (2:3/EtOAc:Hexanes, R_{f} 0.68). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 15a (118 mg, 60%) as a light yellow solid: ¹H NMR (600 MHz, (CD₃)₂SO) a 10.31 (s, 1H), 8.19 (s, 1H), 7.91 (dd, J₁=1.7 Hz, J₂ 0.8 Hz, 1H), 7.25 (dd, J₁=3.5 Hz, J₂=0.8 Hz, 1H), 7.16 (td, J₁=8.2 Hz, J₂=6.7 Hz, 1H), 6.67 (dd, J₁=3.6 Hz, J₂=1.8 Hz, 1H), 6.56 (ddd, J₁=8.2 Hz, J₂=2.2 Hz, J₃=0.8 Hz, 1H), 6.49 (ddd, J₁=10.6 Hz, J₂=8.2 Hz, J₃=2.3 Hz, 1H), 6.45 (dt, J₁=11.6 Hz, J₂=2.3 Hz, 1H); ¹³C NMR (150 MHz, (CD₃)₂SO) δ 164.4 and 162.8 (d. J=238.4 Hz), 158.4, 152.1 and 152.0 (d, J=10.1 Hz), 146.8, 146.2, 130.9 and 130.8 (d, J=9.8 Hz), 114.8, 112.3, 108.6, 105.2 and 105.1 (d, J=21.6 Hz), 99.2 and 99.0 (d, J=25.1 Hz); HRMS m/z calcd for C₁₁H₉FN₂O₂ [M+H]⁺: 221.0726; found 221.0713; Purity of the compound was further confirmed by HPLC: Rₜ=14.83 min (98% pure).

### Example 44: Synthesis of Compound 15e (SGT1819)

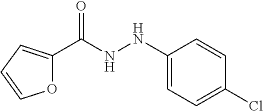 [SMILES: O=C(NNc1ccc(Cl)cc1)c1ccco1]

To a solution of 2-furoic acid (100 mg. 0.89 mmol) in DMF (3 mL) at 0° C. N-(3-dimethylaminopropyl)-N′-ethylcarbodiimide hydrochloride (222 mg, 1.16 mmol), 1-hydroxybenzotriazole hydrate (176 mg, 1.16 mmol), and N,N-diisopropylethyl amine (0.47 mL, 2.67 mmol) were added. The reaction mixture was stirred at 0° C. for 30 min followed by the addition of 4-chlorophenylhydrazine hydrochloride (208 mg, 1.16 mmol). The reaction mixture was stirred at room temperature overnight, and progress of the reaction was monitored by TLC (2:3/EtOAc:Hexanes, R_{f} 0.41). The reaction mixture was quenched with H₂O (80 mL), extracted with EtOAc (80 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 15e (141 mg, 67%) as a yellow solid: ¹H NMR (500 MHz, (CD₃)₂SO) δ 10.31 (br s, 1H), 8.08 (br s, 1H), 7.90 (br s, 1H), 7.24 (d, J=3.5 Hz, 1H), 7.18 (d, J=8.7 Hz, 2H), 6.73 (d, J=8.8 Hz, 2H), 6.67 (dd, J₁=3.6 Hz, J₂=1.8 Hz, 1H); 13C NMR (150 MHz, (CD₃)₂SO) 6158.0, 148.3, 146.4, 145.7, 128.6 (2C), 121.9, 114.2, 113.7 (2C), 111.8; HRMS m/z calcd for C₁₁H₉ClN₂O₂ [M+H]⁺: 237.0431; found 237.0426; Purity of the compound was further confirmed by HPLC: Rₜ=15.39 min (98% pure).

### Example 45: Synthesis of Compound 16a (SGT1883)

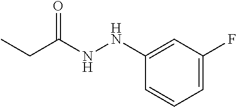

To a solution of propionic acid (0.10 mL, 1.35 mmol) in DMF (3 mL) at 0° C., N-(3-dimethylaminopropyl)-N′-ethylcarbodiimide hydrochloride (336 mg, 1.75 mmol), 1-hydroxybenzotriazole hydrate (237 mg, 1.75 mmol), and N,N-diisopropylethyl amine (0.74 mL, 4.05 mmol) were added. The reaction mixture was stirred at 0° C. for 30 min followed by the addition of 3-fluorophenylhydrazine hydrochloride (285 mg, 1.75 mmol). The reaction mixture was stirred at room temperature overnight, and progress of the reaction was monitored by TLC (2:3/EtOAc:Hexanes, R_{f} 0.61). The reaction mixture was quenched with H₂O (50 mL), extracted with EtOAc (50 mL), washed with additional H₂O (50 mL) and brine (25 mL), and dried over MgSO₄. The organic layer was removed under reduced pressure, and the residue was purified by flash column chromatography (SiO₂, 2:3/EtOAc:Hexanes) to yield compound 16a (159 mg, 87%) as a yellow solid: ¹H NMR (600 MHz, (CD₃)₂SO) δ 9.61 (d, J=2.6 Hz, 1H), 7.96 (d, J=2.5 Hz, 1H), 7.13 (td, J₁=8.1 Hz, J₂=6.8 Hz, 1H), 6.51 (dd, J₁=8.0 Hz, 12=2.0 Hz, 1H), 6.45 (td, J₁=8.5 Hz, 12=2.5 Hz, 1H), 6.40 (dt, J₁=11.7 Hz, J₂=2.3 Hz, 1H), 2.17 (q, J=7.6 Hz, 2H), 1.05 (t, J=7.6 Hz, 3H); ¹³C NMR (150 MHz, (CD₃)₂SO) δ 173.3, 164.5 and 162.9 (d, J=239.2 Hz), 152.25 and 152.18 (d. J=10.7 Hz). 130.8 and 130.7 (d, J=10.3 Hz), 108.5, 104.9 and 104.7 (d, J=20.8 Hz), 99.0 and 98.8 (d, J=25.5 Hz), 27.0, 10.2; HRMS m/z calcd for C₉H₁₁FN₂O [M+H]⁺: 183.0933; found 183.0922; Purity of the compound was further confirmed by HPLC: Rₜ=14.36 min (97% pure).

### Example 46: Synthesis of Compound 16e (SGT1820)

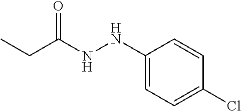

To a solution of propionic acid (100 mg, 1.35 mmol) in DMF (3 mL) at 0° C., N-(3-dimethylaminopropyl)-N′-ethylcarbodiimide hydrochloride (335 mg, 1.75 mmol), 1-hydroxybenzotriazole hydrate (101 mg, 1.75 mmol), and N,N-diisopropylethyl amine (0.71 mL, 4.05 mmol) were added. The reaction mixture was stirred at 0° C. for 30 min followed by the addition of 4-chlorophenylhydrazine hydrochloride (314 mg, 1.75 mmol). The reaction mixture was stirred at room temperature for 12 h, and progress of the reaction was monitored by TLC (2:3/EtOAc:Hexanes, R_{f} 0.21). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 1:1/EtOAc:Hexanes) to afford compound 16e (83 mg, 31%) as a pale yellow solid: ¹H NMR (500 MHz, (CD₃)₂SO) δ 9.60 (d, J=2.6 Hz, 1H), 7.84 (d, J=2.5 Hz, 1H), 7.15 (d, J=8.8 Hz, 2H), 6.68 (d, J=8.8 Hz, 2H), 2.16 (q, J=7.6 Hz, 2H), 1.05 (t, J=7.6 Hz, 3H); ¹³C NMR (150 MHz, (CD₃)₂SO) δ 172.8, 148.5, 128.5 (2C), 121.6, 113.5 (2C), 26.6, 9.8; HRMS n z calcd for C₉H₁₁ClN₂O [M+H]⁺: 199.0638; found 199.0630; Purity of the compound was further confirmed by HPLC: Rₜ=14.90 min (99% pure).

### Example 47: Synthesis of Compound 17e (SGT1821)

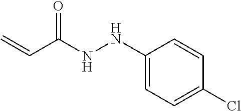

To a solution of acrylic acid (100 mg, 1.39 mmol) in DMF (3 mL) at 0° C., N-(3-dimethylaminopropyl)-N′-ethylcarbodiimide hydrochloride (345 mg, 1.80 mmol), 1-hydroxybenzotriazole hydrate (243 mg, 1.80 mmol), and N,N-diisopropylethyl amine (0.73 mL, 4.17 mmol) were added. The reaction mixture was stirred at 0° C. for 30 min followed by the addition of 4-chlorophenylhydrazine hydrochloride (322 mg, 1.80 mmol). The reaction mixture was stirred at room temperature for 12 h, and progress of the reaction was monitored by TLC (2:3/EtOAc:Hexanes, R_{f} 0.23). The reaction mixture was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 1:1/EtOAc:Hexanes) to afford compound 17e (142 mg, 52%) as a pale yellow solid: ¹H NMR (500 MHz, (CD₃)₂SO) δ 9.95 (d, J=2.8 Hz, 1H), 8.02 (d, J=2.7 Hz, 1H), 7.17 (d, J=8.8 Hz, 2H), 6.69 (d, J=8.8 Hz, 2H), 6.30 (dd, J₁=17.2 Hz, J₂=10.2 Hz, 1H), 6.19 (dd, J₁=17.2 Hz, J₂=2.1 Hz, 1H), 5.72 (dd, J₁=10.2 Hz, J₂=2.1 Hz, 1H); ¹3C NMR (150 MHz, (CD₃)₂SO) δ 164.6, 148.1, 129.5, 128.5 (2C), 126.6, 121.9, 113.6 (2C); HRMS m/z calcd for C₉H₉ClN₂O [M+H]⁺: 197.0481; found 197.0470; Purity of the compound was further confirmed by HPLC: Rₜ=14.89 min (96% pure).

### Example 48: Biological Assay—Bacterial Strains

All synthesized compounds were first tested against several Gram-negative and Gram-positive bacterial strains to assess their activity. These strains were obtained from different sources. Klebsiella pneumoniae ATCC 27736 (strain F), Salmonella enterica ATCC 14028 (strain H), and Escherichia coli MC1061 (strain D) were kindly provided by Prof Paul J. Hergenrother (University of Illinois at Urbana-Champaign, Champaign, IL, USA). Acinetobacter baumannii ATCC 19606 (strain I) was purchased from the American Type Culture Collection (ATCC, Manassas, VA, USA). A. baumannii ATCC BAA1605 (strain J) was generously provided by Prof. Ryan Altman (Purdue University, West Lafayette, IN, USA). Bacillus anthracis 3452 str. Sterne (strain A) came from Prof. Philip C. Hannah (University of Michigan, Ann Arbor, MI, USA). Enterobacter cloacae ATCC 13880 (strain E), Pseudomonas aeruginosa ATCC 14028 (strain G), Staphylococcus aureus ATCC 25923 (strain B), and Staphylococcus epidermidis ATCC 12228 (strain C) were a generous gift from Prof Dev P. Arya (Clemson University, Clemson. SC, USA). All remaining A. baumannii strains were obtained from the CDC and FDA antibiotic resistance (AR) isolate bank.

### Example 49: Biological Assay—Determination of Minimum Inhibitory Concentration (MIC) Values

All synthesized compounds and ciprofloxacin (CIP) were dissolved in DMSO (final stock concentration of 10 mM, 5 mM for CIP). All bacterial strains listed above were stored at −80° C. All strains were streaked on Mueller-Hinton (MH) plates and allowed to grow at 37° C. overnight. Enough colonies were scraped from the plate and suspended in MH broth with vortexing to reach an attenuance at 600 nm (OD₆₀₀) of ˜0.5. The resulting culture was diluted 1:1000 and added to the prepared serially doubly diluted compound solutions in 96-well plates. For M. smegmatis, a 1:100 dilution of the bacteria was done in MH. All MIC values were determined using 96-well plates with low evaporation seals at least in duplicate. For each row, one well was reserved for sterile control (which consisted of 200 μL of MH medium) and another well was reserved for growth control (which consisted of 100 μL of MH medium seeded with 100 μL of bacterial culture). Select MIC data are presented in Tables below (See Example 56).

### Example 50: Biological Assay—Time-Kill Curves

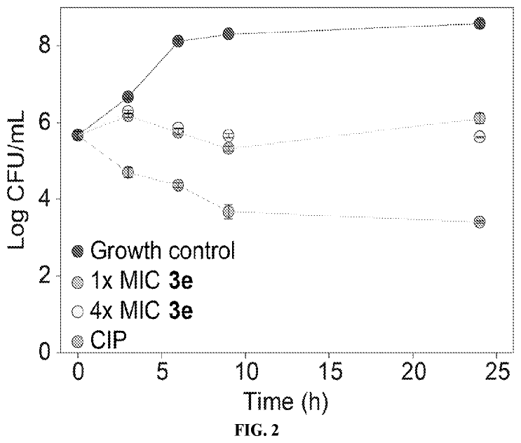

Compound 3e was shown to be effective against many strains of A. baumannii. The killing efficiency of 3e against strain A. baumannii ATCC 19606 (strain I) was assessed by following the time-kill curve procedure as reported with slight modifications.¹ Cells were grown the same as described above. Compound 3e and CIP (positive control) were used at concentrations of 1× and 4× of their respective MIC values. At 0, 3, 6, 9, and 24 h. 100 μL were aliquoted out from the culture and serially diluted in sterile MH medium. 100 μL of serially diluted samples were then spread onto MH plate and incubated at 37° C. for 24 h. The experiments were carried out in duplicate. Colonies were counted to estimate the number of cells in the experiment. The time-kill data are presented in FIG. 2 (See Example 58).

### Example 51: Biological Assay—Biofilm Disruption Assays

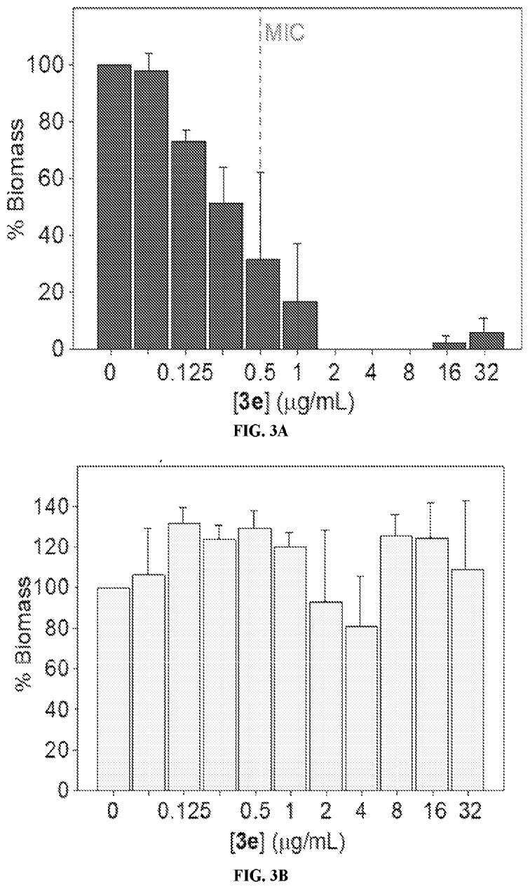

The ability of compounds 3e and CIP to disrupt biofilms was tested based on a previously published protocol. Briefly. A. baumannii ATCC 19606 (strain I) was grown overnight in Tryptic Soy Broth (TSB) at 37° C. The bacteria were then diluted 1:100 in TSB with 1% glucose and plated (100 μL) in 96-well plates. The cells were grown overnight at 37° C. before the medium and planktonic bacteria were removed and the biofilm washed with phosphate buffered saline (PBS; 4×100 μL). At this point, compounds 3e and CIP, dissolved in TSB (100 μL), were added to the biofilms and incubated overnight at 37° C. The compounds and medium were removed by submerging the entire plate in H2O. The biofilms were stained with 100 μL of a 0.1% crystal violet solution by incubating 100 μL of the solution in the wells for 30 min. The excess dye was removed by washing with ddH2O (4×100 μL). The plates were dried for 1 h and the dye was solubilized with 95% EtOH. After thorough mixing the absorbance at 595 nm was read on a SpectraMax M5 plate reader. All readings were normalized to the biofilm formation without compound. The data for biofilm disruption are presented in FIG. 3B (See Example 58).

### Example 52: Biological Assay—Biofilm Prevention Assays

The activity of the compounds in preventing biofilm formation by A. baumannii ATCC 19606 (strain I) was tested similarly to with the disruption assay, except that the compounds were added before the formation of the biofilm. The data for prevention of biofilm formation are presented in FIG. 3A (See Example 58).

### Example 53: Biological Assay—Mammalian Cytotoxicity Assays

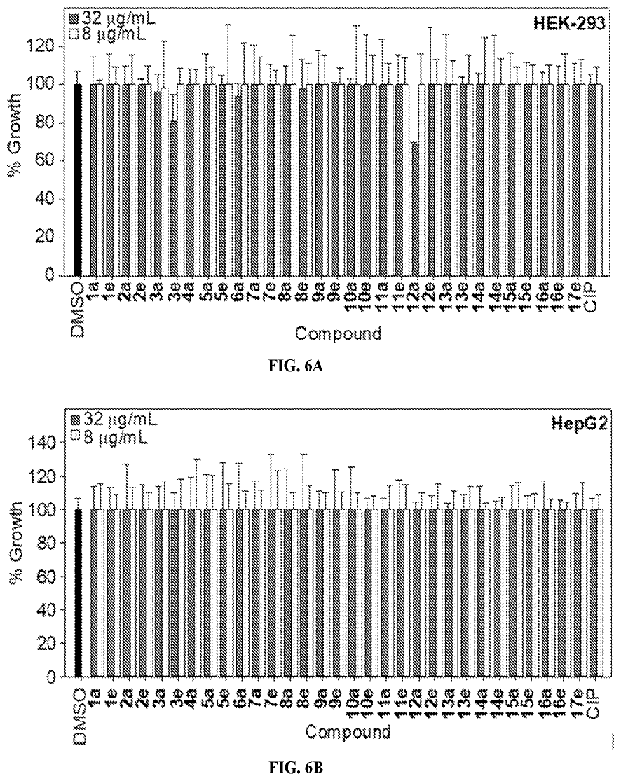

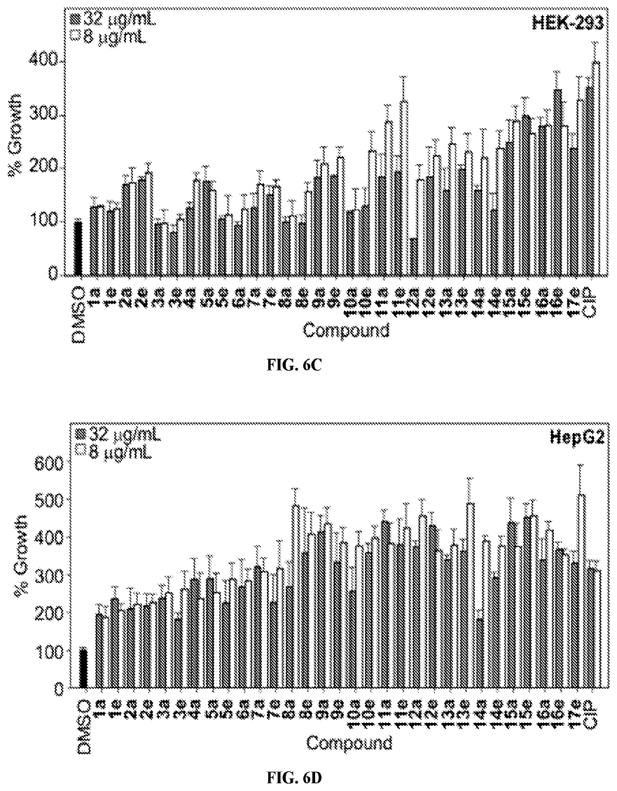

The cytotoxicity against mammalian cells was measured based on a previously published protocol² using human embryonic kidney (HEK-293), and epithelial-like morphology hepatocellular carcinoma (HepG2). The cells were cultured in Dulbecco's Modified Eagle's Medium (DMEM) supplemented with 10% fetal bovine serum (FBS) and 1% penicillin/streptomycin at 37° C. with 5% CO2 until confluent. Cells at a concentration of 10,000 cells/mL were plated in 96-well plate (100 μL) and grown overnight. The next day, the medium was replaced with 100 μL of fresh DMEM and 100 μL of DMEM containing test compounds at the final concentrations of 32 or 8 μg/mL. The cells were incubated with tested compounds for another 24 h at 37° C. with 5% CO2. Cell survival was assessed by a resazurin assay, as follows. Each well was treated with resazurin (10 μL of a 2 mM solution) for 6 h. Live cells produced a pink and the highly fluorescent dye, resorufin, that was detected by using a SpectraMax M5 plate reader to quantify the viability of the cells. These data are presented in FIG. 6A-D (See Example 60).

### Example 54: Biological Assay—Hemolysis Assays

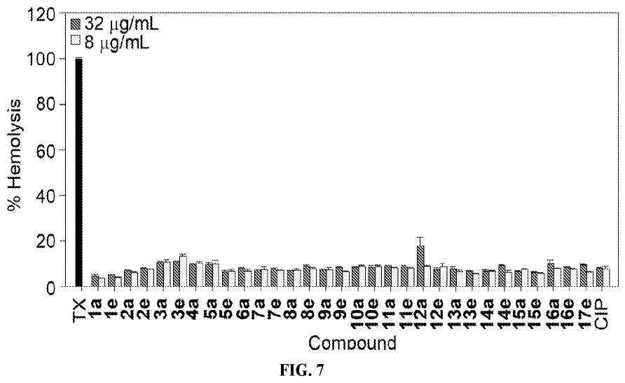

Hemolytic activity of compounds 1a, 1e, 2a, 2e, 3a, 3e, 4a, 5a, 5e, 6a, 7a, 7e, 8a, 8e, 9a, 9e, 10a, 10e, 1a, 1e, 12a, 12e, 13a, 13e, 14a, 14e, 15a, 15e, 16a, 16e, 17e, and CIP was assessed using methods previously reported with minor modifications.³ The citrate-treated murine red blood cells (mRBCs; 1 mL) were suspended in 8 mL of sterile phosphate buffered saline (PBS). The cells were then washed using PBS (3×) by spinning at 1,000 rpm for 7 min. After washing, mRBCs were counted using a hemacytometer and diluted in PBS to 5×10⁷ cells/mL. All the tested compounds were diluted (32 μg/mL and 8 μg/mL) in PBS in Eppendorf tubes (500 μL volume) and 100 μL were added to each of the columns of the 96-well plate except for column 1. A positive control (1% Triton-X®, TX), a negative control (DMSO), and wells with no mRBCs to account for any background absorbance were used in the 96-well plate. After the addition of the compounds to the plate, 100 μL of mRBCs were added to each well and plates were incubated at 37° C. for 30 min. The plates were then spun at 1,000 rpm for 10 min till the mRBCs settle at the bottom of the plates. 100 μL of supernatant was then transferred to a new 96-well plate and the absorbance at 500 nm was read using a spectrophotometer (SpectraMax M5). Percent hemolysis was calculated using the following equation after subtraction of the background absorbance. These data are presented in FIG. 7 (See Example 61).

$$\% hemolysis = \frac{{Abs}_{500} of compound}{{Abs}_{500} of TX} \times 100$$

### Example 55: Biological Assay—Development of Resistance

To determine if the bacteria would become resistant to the compounds, 15 passages of MIC using A. baumannii ATCC 19606 (strain I) were carried out as above (see above). After each MIC experiment, the bacteria at ½×MIC were grown and used in the next MIC experiment (FIG. 5; See Example 59).

### Example 56: The High Anti-Ab Potency of these Compounds and their High Selectivity Against Ab

Minimum inhibitory concentrations (MIC) of all compounds from the series were measured in liquid cultures of different Ab strains by a double-dilution method in Mueller-Hinton broth. Initially, the activity of all 46 compounds against Ab ATCC 19606 (a control strain) and Ab ATCC BAA1605 (a multidrug-resistant strain) (Table 1; data shown for 27 representative compounds) was measured. The main structure-activity relationships that emerged from these MIC data included (i) the fact that all compounds with R₂═(CH₂CO₂Et (g) and R₂=cyclohexyl (h) were completely inactive against Ab, (ii) compounds with R₂=3-F-Ph (a) and R2=4-Cl-Ph (e) were highly active, and (iii) all R1 groups (1-15) tested resulted in active compounds, indicating the importance of the aromatic group on the hydrazine side of the molecules.

TABLE 1
MIC values (μg/mL) for 27 representative monohydrazide compounds
and control kanamycin (KAN) against a control Ab strain ATCC
19606 and a multidrug-resistant strain ATCC BAA1605.

Strain

| Compound | Ab ATCC 19606 | Ab ATCC BAA 1605 |
|---|---|---|
| 1e | 0.25 | 0.25 |
| 2a | 2 | 0.5 |
| 2b | 2 | <0.5 |
| 2c | 0.5 | 1 |
| 2e | 0.25 | 0.25 |
| 3a | 0.5 | 0.5 |
| 3c | 0.5 | 0.5 |
| 3d | 0.5 | 0.25 |
| 3e | 1 | 0.0625 |
| 3f | 1 | 0.5 |
| 3g | >64 | >64 |
| 4a | 0.5 | 0.5 |
| 5e | 0.125 | 0.125 |
| 5g | >64 | >64 |
| 6a | 0.5 | 0.5 |
| 7e | 0.125 | 0.125 |
| 8e | 0.125 | 0.0625 |
| 9a | 0.5 | 0.5 |
| 10a | 0.25 | 0.5 |
| 11a | 0.25 | 0.5 |
| 11h | >64 | >64 |
| 12e | 0.5 | 0.5 |
| 13e | 0.5 | 0.25 |
| 14a | 0.25 | 0.25 |
| 15e | 0.5 | ≤0.125 |
| 16e | ≤0.25 | ≤0.125 |
| 17e | 0.25 | 0.25 |
| KAN | 5 | 10 |

Then all 46 compounds were tested against a broad panel of all 41 Ab strains from the CDC and FDA Antibiotic Resistance (AR) Isolate Bank (Table 2; data shown for 2 representative compounds), which were all highly multidrug-resistant strains, including those resistant to colistin. Regardless of their R1 groups (145), compounds with R₂ (a-f) were highly potent against all 41 strains tested, with some MIC values as low as 0.03 (μg/mL), or ˜100 nM. Those with R₂ (g or h) were inactive. The activity of the monohydrazides was not dependent on the extent (number of resistance mutations) or severity (antibiotic MIC) of resistance. All conclusions made from Table 1 remained valid for all compounds against these 41 additional Ab strains. Taken together with the fact that the molecules belong to a yet unexplored structural class, these data strongly suggest that these monohydrazides act by a novel mechanism of action. This mechanism of action will be elucidated in the proposed studies.

TABLE 2
MIC values (μg/mL) for 2 representative monohydrazide compounds (3e
and 9a) against all 41 multidrug-resistant Ab strains (#273-313) from
the CDC and FDA AR Bank wwwn.cdc.giv/ARIsolateBank/Panel/PanelDetail?ID=1).

| Compound | 273 | 274 | 275 | 276 | 277 | 278 | 279 | 280 | 281 | 282 |
|---|---|---|---|---|---|---|---|---|---|---|
| 3e | 0.5 | 0.5 | 0.25 | 0.03 | 0.06 | 0.25 | 0.25 | 0.5 | 0.5 | 0.25 |
| 9a | 1 | 1 | 0.5 | 0.5 | 1 | 1 | 1 | 1 | 0.5 | 1 |
| Compound | 283 | 284 | 285 | 286 | 287 | 288 | 289 | 290 | 291 | 292 |
| 3e | 0.5 | 0.25 | 0.13 | 0.06 | 0.13 | 0.25 | 0.25 | 0.125 | 0.06 | 0.25 |
| 9a | 0.25 | 0.5 | 0.5 | 1 | 0.5 | 1 | 0.5 | 1 | 0.25 | 0.25 |

| Compound | 293 | 294 | 295 | 296 | 297 | 298 | 299 | 300 | 301 | 302 |
|---|---|---|---|---|---|---|---|---|---|---|
| 3e | 0.13 | 0.25 | 0.06 | 0.06 | 0.25 | 0.25 | 0.13 | 0.25 | 0.25 | 0.13 |
| 9a | 0.25 | ≤0.13 | ≤0.13 | ≤0.13 | 0.5 | 0.5 | 0.25 | 0.25 | 0.25 | 0.25 |

| Compound | 303 | 304 | 305 | 306 | 307 | 308 | 309 | 310 | 311 | 312 |
|---|---|---|---|---|---|---|---|---|---|---|
| 3e | 0.13 | 0.5 | 0.5 | 0.13 | 0.5 | 0.13 | 0.25 | 0.25 | 0.5 | 0.5 |
| 9a | 0.25 | ≤0.13 | 0.5 | 0.5 | 0.5 | 0.25 | 0.5 | 0.5 | 0.5 | 0.5 |

| Compound | 313 |
|---|---|
| 3e | 0.25 |
| 9a | 0.25 |

Note:
control KAN had MIC values of >10 μg/mL against almost all strains.

Next, to address the selectivity of these compounds and their antibacterial spectrum, some of the active monohydrazides listed in Table 1 were tested against a panel of several different non-Ab bacterial species. Most of the compounds were inactive against all the bacterial strains tested. Only a small subset of the compounds (shown in Tables 3 and 4) had MIC values in the range of 4-8 μg/mL against one or a few bacteria (for example, the MIC of 2e against Mycobacterium smegmatis was 4 μg/mL). However, even these values were much higher than the respective MIC values against Ab strains. These data indicate the narrow antibacterial spectrum of the monohydrazides, and that the target of these molecules is likely present, or most accessible, only in Ab. An alternative explanation to the narrow spectrum is that these compounds act as Ab-selective prodrugs after getting chemically converted to an active agent by Ab and not by other bacteria.

TABLE 3
MIC values (μg/mL) for monohydrazides against some non-Ab bacteria
(set 1). Ab ATCC BAA1605 was used as a positive control (last column).

| | K. pneumoniae | P. aeruginosa | E. coli ATCC | K. pneumoniae | A. Baumannii |
|---|---|---|---|---|---|
| Compound | ATCC 27736 | ATCC 27853 | BAA 2469 | ATCC BAA 1705 | ATCC BAA 1605 |
| 2b | 8 | >128 | 8 | 16 | ≤0.5 |
| 2c | >128 | >128 | >128 | >128 | 1 |
| KAN | 4 | >128 | >128 | >128 | 32 |

TABLE 4
MIC values (μg/mL) for monohydrazides against some non-Ab bacteria (set 2).

| | B. | S. enterica | E. cloacae | | S. Aureus | Y. | |
|---|---|---|---|---|---|---|---|
| | anthracis | ATCC | ATCC | S. epidermidis | ATCC | pestis | M. |
| Compound | Sterne F32 | 14028 | 13047 | ATCC 12228 | 25923 | CO92 | smegmatis |
| 2e | >16 | >16 | >16 | >16 | >16 | 16 | 4 |
| 3e | >16 | 8 | 4 | >16 | >16 | 8 | 4 |
| KAN | >160 | 2.5 | 1.25 | 0.63 | 0.63 | 2.5 | ≤0.03 |

### Example 57: Antibacterial Activity

The compounds were tested against a panel of bacteria containing both Gram-positive and Gram-negative bacteria. Initially, Bacillus anthracis F32 Sterne (strain A), Staphylococcus aureus ATCC 25923 (strain B), Staphylococcus epidermidis ATCC 12228 (strain C), Escherichia coli MC1061 (strain D), Enterobacter cloacae ATCC 13047 (strain E), Klebsiella pneumoniae ATCC 27736, (strain F), Pseudomonas aeruginosa ATCC 14028 (strain G), Salmonella enterica ATCC 14028, (strain H), and Acinetobacter baumannii ATCC 19606 (strain I) were used with a set of structurally varied compounds and ciprofloxacin (CIP) as a control antibiotic. The hydrazide compounds were inactive or very weakly active against the Gram-positive bacteria with inhibition of growth occurring at >64 μg/mL (Table 1). Several compounds (1e, 2b, 5e, 10e, and 15e-17e) had moderate activity against the Gram-negative bacteria, with MIC values in the range 4-16 μg/mL. Surprisingly, all the initially tested compounds, except for 12 g and 12 h, were as active or better than the CIP control in inhibiting cell growth of A. baumannii ATCC 19606 (strain I), with some MIC values as low as 125 ng/mL or lower. To understand if this effect was strain or species specific, a second strain of A. baumannii, ATCC BAA1605 (strain J) was tested. All compounds had comparable MIC values for both strains of A. baumannii. Compound 3e seemed to be much better at inhibiting the growth of strain J over strain I (Table 5).

TABLE 5
MIC values (in μg/mL) determined in Mueller-Hinton medium for selected synthesized
compounds and for the control antibacterial drug CIP against various bacterial strains.

| | Gram-(+)ve | | | Gram-(−)ve | | | | | | |
|---|---|---|---|---|---|---|---|---|---|---|
| Cpd # | A | B | C | D | E | F | G | H | I | J |
| 1e | >64 | >64 | >64 | 16 | 16 | >64 | >64 | >64 | 0.25 | 0.25 |
| 2b | >64 | >64 | >64 | 4 | 4 | 16 | >64 | 16 | 2 | 1 |
| 2e | >64 | >64 | >64 | 16 | 64 | 64 | >64 | 64 | 0.25 | 0.25 |
| 3a | >64 | >64 | >64 | 64 | >64 | >64 | >64 | >64 | 0.5 | 0.5 |
| 3c | >64 | >64 | 64 | >64 | >64 | >64 | >64 | >64 | 0.5 | 0.5 |
| 3d | >64 | >64 | >64 | 64 | 64 | 64 | >64 | 64 | 0.5 | 0.25 |
| 3e | >64 | >64 | >64 | 16 | 16 | 64 | >64 | 64 | 1 | ≤0.125 |
| 3f | >64 | >64 | >64 | >64 | >64 | >64 | >64 | >64 | 1 | 0.5 |
| 5e | >64 | >64 | >64 | 16 | 16 | 16 | >64 | 16 | ≤0.125 | ≤0.125 |
| 10e | >64 | 64 | 64 | 16 | 16 | 16 | >64 | 64 | 0.25 | 0.5 |
| 12e | >64 | >64 | >64 | 16 | >64 | >64 | >64 | >64 | 0.5 | 0.5 |
| 12g | >64 | >64 | >64 | >64 | >64 | >64 | >64 | >64 | >64 | >64 |
| 12h | >64 | >64 | >64 | 64 | >64 | >64 | >64 | >64 | >64 | >64 |
| 14e | >64 | >64 | >64 | >64 | >64 | >64 | >64 | >64 | 0.5 | 0.25 |
| 15e | >64 | 64 | 64 | 16 | 4 | 16 | >64 | 16 | 0.5 | ≤0.125 |
| 16e | >64 | >64 | >64 | 16 | 16 | 16 | >64 | 64 | ≤0.125 | ≤0.125 |
| 17e | >64 | 64 | 64 | 16 | 16 | 16 | >64 | 16 | 0.25 | 0.25 |
| CIP | 1 | ≤0.0625 | ≤0.0625 | ≤0.0625 | ≤0.0625 | ≤0.0625 | 0.25 | ≤0.0625 | 1 | 2 |

Bacterial strains: A = B. anthracis Sterne F32, B = S. aureus ATCC 25923, C = S. epidermidis ATCC 12228, D = E. coli MC1061, E = E. cloacae ATCC 13047, F = K. pneumoniae ATCC 27736, G = P. aeruginosa ATCC 27853, H = S. enterica ATCC 14028, I = A. baumannii ATCC 19606, J = A. baumannii BAA1605.
CIP = ciprofloxacin.

Having established that the observed activity is selective to A. baumannii, all synthesized compounds were tested against a panel of antibiotic resistant A. baumannii from the Antimicrobial Resistant (AR) Isolate Bank from the CDC along with strains I and J (Table Si). The MIC value statistics for all 43 strains of A. baumannii (41 CDC strains and 2 ATCC strains) are summarized in Table 6. The compounds containing an aryl group on the hydrazine functionality were highly active against all the tested A. baumannii strains, whereas the compounds lacking an aryl group on the hydrazine functionality (R₂, g and h) failed to visibly affect the growth of the A. baumannii strains. Compounds 3e, 5e, 10e, 15e, and 16e, had MIC values of <0.125 μg/mL for 13-20 strains each (30-46% of all strains). Compounds 1e, 2e, 3a, 7e, 8e, 10a, 12e, and 17e had MIC values of <0.125 g/mL for 5-9 strains each (11-21% of all strains). All remaining compounds inhibited 0-3 (<7%) strains with an MIC value of 0.125 μg/mL. The mode of the data set for the MIC values of these compounds with the A. baumannii strains is 0.5 μg/mL. Overall, the best compounds were those with an “a” or “e” hydrazine. Based on this statistical analysis, the best compound is 5e followed closely by 3e.

TABLE 6
Summary of MIC values (in μg/mL) determined for all
A. baumannii strains tested with the synthesized
compounds. Numbers indicate the number of compound/strain
combinations with the corresponding MIC value.

| | MIC value (μg/mL) | | | | | | | |
|---|---|---|---|---|---|---|---|---|
| Cpd # | ≤0.125 | 0.25 | 0.5 | 1 | 2 | 4 | 8 | >64 |
| 1a | 1 | 3 | 26 | 9 | 4 | | | |
| 1e | 8 | 17 | 12 | 5 | 1 | | | |
| 1g | | | | | | | | 43 |
| 1h | | | | | | | | 43 |
| 2a | 2 | 10 | 18 | 10 | 3 | | | |
| 2b | 2 | 13 | 13 | 12 | 3 | | | |
| 2c | 1 | 5 | 6 | 28 | 3 | | | |
| 2e | 5 | 16 | 21 | 1 | | | | |
| 2g | | | | | | | | 43 |
| 3a | 9 | 16 | 18 | | | | | |
| 3c | 1 | 2 | 12 | 23 | 5 | | | |
| 3d | 1 | 10 | 19 | 13 | | | | |
| 3e | 16 | 16 | 10 | 1 | | | | |
| 3f | | 1 | 2 | 9 | 19 | 11 | 1 | |
| 3g | | | | | | | | 43 |
| 4a | | 2 | 8 | 19 | 14 | | | |
| 5a | | 1 | 8 | 25 | 9 | | | |
| 5e | 20 | 21 | 2 | | | | | |
| 5g | | | | | | | | 43 |
| 6a | | 4 | 16 | 19 | 4 | | | |
| 7a | 1 | 5 | 19 | 18 | | | | |
| 7e | 8 | 28 | 7 | | | | | |
| 8a | 2 | 11 | 12 | 18 | | | | |
| 8e | 7 | 34 | 2 | | | | | |
| 9a | 4 | 11 | 18 | 10 | | | | |
| 9e | 1 | 8 | 19 | 15 | | | | |
| 9g | | | | | | | | 43 |
| 9h | | | | | | | | 43 |
| 10a | 6 | 19 | 17 | 1 | | | | |
| 10e | 13 | 23 | 6 | 1 | | | | |
| 11a | 2 | 11 | 26 | 4 | | | | |
| 11e | 5 | 15 | 19 | 4 | | | | |
| 11h | | | | | | | | 43 |
| 12a | | 8 | 21 | 14 | | | | |
| 12e | | 7 | 19 | 17 | | | | |
| 12g | | | | | | | | 43 |
| 12h | | | | | | | | 43 |
| 13a | | 6 | 19 | 15 | 3 | | | |
| 13e | | 9 | 22 | 12 | | | | |
| 14a | | 13 | 25 | 5 | | | | |
| 14e | 3 | 18 | 20 | 2 | | | | |
| 15a | 6 | 21 | 16 | | | | | |
| 15e | 13 | 18 | 10 | 2 | | | | |
| 16a | 4 | 10 | 15 | 14 | | | | |
| 16e | 15 | 17 | 10 | 1 | | | | |
| 17e | 9 | 21 | 13 | | | | | |
| Total | 163 | 447 | 524 | 306 | 93 | 14 | 1 | 473 |
| %^{a} | 10.6 | 28.8 | 33.9 | 19.7 | 6.0 | 0.9 | 0.1 | — |

^{a}Not including g and h compounds (>64 μg/mL only compounds).

Over the past several years, multiple reports of anti-A. baumannii compounds have been published. Two reports by Alam and coworkers studied the effect of substituted pyrazoles on Gram-negative bacteria.^{27, 28} These studies investigated the effect of pyrazoles substituted with at least three aromatic rings. In both cases, the compounds were equal to the control antibiotic or had inhibitory values 3- to 4-fold worse than the control. The target of these molecules is currently being investigated. Oxidative phosphorylation inhibitors, 1,2- and 1,3-disubstituded 2-amino benzimidazole derivatives, were reported in 2015.²⁹ These compounds were shown to inhibit A. baumannii membrane ATP synthesis, however, the MIC values of these compounds ranged from 13 to >100 μM. A series of N-carboxy pyrrolidines, potential enoyl reductase inhibitors, were also tested against A. baumannii.³⁰ These compounds turned out to be better suited for Gram-positive strains. In 2017, a virtual screening for compounds binding to the outer membrane protein W2 of A. baumannii led to the discovery of two phenylhydrazides, structurally distinct from those presented here, with anti-A. baumannii activity in the 1-32 μg/mL range (strain-dependent).³′ These compounds were found to be bacteriostatic. Most compounds presented herein have better MIC values against A. baumannii than those previously published by other groups.

### Example 58: Compounds are Bacteriostatic and Inhibit Cell Growth Both in Liquid Culture and as Biofilms

To determine whether the monohydrazides are bacteriostatic or bactericidal agents, the killing efficiency of one of the active compounds, 3e, was assessed against ciprofloxacin-susceptible control strain Ab ATCC 19606. A previously reported method was used,¹³ with minor modifications. Briefly, compound 3e was added at 1′ MIC and 4′ MIC to the Ab bacteria grown in liquid broth. Then at specified incubation times (FIG. 2), bacteria were serially diluted, plated on agar plates not containing the compound, and incubated overnight, to obtain the colony count. The data (FIG. 2) showed that upon incubation at compound concentrations 1×MIC and 4×MIC, there were no additional viable bacteria produced and no killing of existing bacteria was observed, indicating that 3e was a bacteriostatic agent. Ciprofloxacin, a bactericidal agent, reduced the colony count 4100-fold, as expected. A novel anti-Ab drug does not need to be bactericidal; minocycline is a bacteriostatic agent that has shown high clinical efficacy as monotherapy against multidrug-resistant (but minocycline-susceptible) Ab.¹⁴

Biofilms are complex communities of one or more species of bacteria and/or fungi encased in a polymeric extracellular substance and attached to surfaces. Because of the complex nature of these coatings and the effect they can have on the environment and local populations of bacteria, antimicrobial agents often have difficulty reaching the targeted pathogens in these networks.³²

A crystal violet assay was used to detect the biomass of the produced biofilm for A. baumannii ATCC 19606 (strain I). Both biofilm prevention and biofilm disruption were monitored by adding the compound of interest, 3e (MIC=0.5 g/mL under conditions used to generate biofilm, either after the formation of biofilm (disruption) or while biofilm is forming (prevention). These assays and the complementary MIC determination were carried out in trypticase soy broth (TSB) medium analogously to a method recently reported.¹⁵

Compound 3e displayed inhibition of biofilm formation consistent with inhibition of bacterial growth (FIG. 3A), as biofilm formation was abolished at concentrations above MIC. Compound 3e did not disrupt a preformed Ab biofilm (FIG. 3B).

### Example 59: Compounds are not Cytotoxic to Mammalian Cells

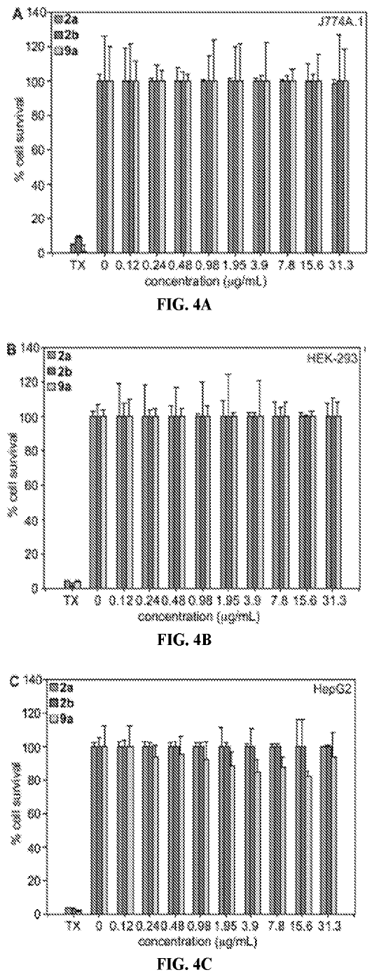

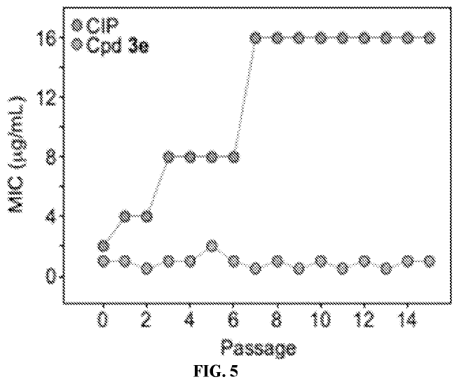

Cytotoxicity of three active compounds (2a, 2b, and 9a) to three mammalian cell lines: mouse macrophages (J774A.1), human embryonic kidney cells (HEK-293), and human hepatocarcinoma (HepG2) was tested, analogously to the previously reported method (FIG. 4). None of the three tested monohydrazides displayed any toxicity against J774A.1 and HEK-293 cells up to the highest concentration used in these assays (31.3 μg/mL). Compounds 2a and 2b were also nontoxic to HepG up to 31.3 μg/mL, whereas 9a exhibited minor toxicity (cell survival >85%) to HepG2 above 1.95 μg/mL. These data also strongly support drug development of this monohydrazide series for treatment of Ab infections.

Ab resistance to the monohydrazides does not develop upon multiple passages. Because Ab is notorious for being able to readily mount antibiotic resistance, Ab ATCC 19606 was tested to determine whether it can develop resistance to potent compound 3e in a multiple passage MIC assay. Fifteen passages of MIC assays were performed, where the bacteria grown at concentration of 3e of ½×MIC is used in the next MIC experiment and so on (FIG. 5). While resistance to the ciprofloxacin (CIP) control started emerging after one passage, from an MIC of 2 μg/mL to 16 g/mL, resistance to 3e was not observed even after 15 passages (FIG. 5). The lack of Ab resistance development upon passaging at sub-MIC levels of 3e is highly encouraging for development of these monohydrazides as anti-Ab drugs.

### Example 60: Cytotoxicity to Mammalian Cells

Having established the excellent activity of the hydrazides against A. baumannii strains, the compounds were tested for toxicity to mammalian cells. The effect of the compounds were examined on human embryonic kidney cells (HEK-293) and human hepatoma cells (HepG2), as many compounds are metabolized in the liver and/or excreted through the kidneys. Compounds from the a and e family were used for this assay, as they had the best overall activity against the A. baumannii strains. None of the compounds showed any toxicity to either cell type at 8 μg/mL, indicating an excellent safety profile. Compound 3e was slightly toxic to HEK-293 cells at 32 g/mL, inhibiting growth by 17%, and compound 12a when used at 32 μg/mL inhibited growth of HEK-293 by 40% (FIG. 6A-D). None of the compounds or CIP showed any cytotoxicity to HepG2 cells.

### Example 61: Hemolytic Activity

To test the safety of the compounds, they were for exampled for hemolytic effect on murine red blood cells (FIG. 7). All compounds displayed less than 10% hemolysis when tested at 8 μg/mL. This effect was like that of the CIP control. The same high safety was observed for all compounds at 32 g/mL, apart from 12a, which exhibited 20% hemolysis. Interestingly, the same compound displayed toxicity to HEK-293 cells, suggesting that the toxicity could be due to membrane disruption. These results indicate that the compounds (except for 12a) are not hemolytic.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Dijkshoorn, L.: Nemec, A.; Seifert, H., An increasing threat in hospitals: Multidrug-resistant Acinetobacter baumannii. Nat. Rev. Microbiol. 2007, 5 (12), 939-951.
2. Tacconelli, E.; Carrara, E.; Savoldi, A.; Harbarth, S.; Mendelson, M.; Monnet, D. L.; Pulcini, C.; Kahlmeter, G.; Kluytmans, J.; Carmeli, Y.; Ouellette, M.; Outterson, K.; Patel, J.; Cavaleri, M.; Cox, E. M.; Houchens, C. R.; Grayson, M. L.; Hansen, P.; Singh, N.; Theuretzbacher. U.; Magrini, N.; Group, W. H. O. P. P. L. W., Discovery, research, and development of new antibiotics: the WHO priority list of antibiotic-resistant bacteria and tuberculosis. Lancet Infect. Dis. 2018, 18 (3), 318-327.
3. Villegas, M. V.; Hartstein, A. I., Acinetobacter outbreaks, 1977-2000. Infect. Control Hosp. Epidemiol. 2003, 24 (4), 284-295.
4. Peleg, A. Y.; Seifert, H.; Paterson, D. L., Acinetobacter baumannii: Emergence of a successful pathogen. Clin. Microbiol. Rev. 2008, 21 (3), 538-582.
5. Falagas, M. E.; Rafailidis, P. I., Attributable mortality of Acinetobacter baumannii: No longer a controversial issue. Crit. Care 2007, 11 (3), 134.
6. Fournier, P. E.; Richet, H., The epidemiology and control of Acinetobacter baumannii in health care facilities. Clin. Infect. Dis. 2006, 42 (5), 692-699.
7. Lin, M. F.; Lan, C. Y., Antimicrobial resistance in Acinetobacter baumannii: From bench to bedside. World J. Clin. Cases 2014, 2, 787-814.
8. Queenan, A. M.; Bush, K., Carbapenemases: The versatile beta-lactamases. Clin. Microbiol. Rev. 2007, 20 (3), 440-458.
9. Kurihara. M. N. L.; Sales, R. O.: Silva, K. E. D.; Maciel, W. G.; Simionatto, S., Multidrug-resistant Acinetobacter baumannii outbreaks: A global problem in healthcare settings. Rev. Soc. Bras. Med. Trop. 2020, 53, e20200248.
10. Piperaki, E. T.; Tzouvelekis, L. S.; Miriagou, V.; Daikos, G. L., Carbapenem-resistant Acinetobacter baumannii: In pursuit of an effective treatment. Clin. Microbiol. Infect. 2019, 25 (8), 951-957.
11. Isler, B.; Doi, Y.; Bonomo, R. A.; Paterson, D. L., New treatment options against carbapenem-resistant Acinetobacter baumannii infections. Antimicrob. Agents Chemother. 2019, 63 (1), e01110-18.
12. Bassetti, M.; Echols. R.; Matsunaga. Y.; Ariyasu, M.; Doi, Y.; Ferrer, R.; Lodise, T. P.; Naas. T.; Niki, Y.; Paterson, D. L.: Portsmouth, S.; Torre-Cisneros, J.; Toyoizumi, K.; Wunderink, R. G.; Nagata, T. D., Efficacy and safety of cefiderocol or best available therapy for the treatment of serious infections caused by carbapenem-resistant Gram-negative bacteria (CREDIBLE-CR): A randomised, open-label, multicentre, pathogen-focused, descriptive, phase 3 trial. Lancet Infect. Dis. 2021, 21 (2), 226-240.
13. Ngo, H. X.; Shrestha, S. K.; Green, K. D.; Gameau-Tsodikova, S., Development of ebsulfur analogues as potent antibacterials against methicillin-resistant Staphylococcus aureus. Bioorg. Med. Chem. 2016, 24 (24), 6298-6306.
14. Lashinskv, J. N.: Henig, O.; Pogue, J. M.; Kaye. K. S., Minocycline for the treatment of multidrug and extensively drug-resistant A. baumannii: A review. Infect. Dis. Ther. 2017, 6 (2), 199-211.
15. Green, K. D.: Punetha, A.; Chandrika, N. T.; Hou, C.; Garneau-Tsodikova, S.; Tsodikov, O. V., Development of single-stranded DNA bisintercalating inhibitors of primase DnaG as antibiotics. ChemMedChem 2021, 16 (12), 1986-1995.
16. Monem, S., Furmanek-Blaszk, B., Lupkowska, A., Kuczvnska-Wisnik, D., Stojowske-Swedrzynska, K., and Laskowska, E. (2020) Mechanisms protecting Acinetobacter baumannii against multiple stresses triggered by the host immune response, antibiotics and outside-host environment, Int. J. Mol. Sci. 21, 5498.
17. Mulani, M. S., Kamble, E. E., Kumkar, S. N., Tawre, M. S., and Pardesi, K. R. (2019) Emerging strategies to combat ESKAPE pathogens in the era of antimicrobial resistance: A review, Front. Microbiol. 10, 539.
18. Guidelines for the prevention and control of carbapenem-resistant Enterobacteriaceae, Acinetobacter baumannii and Pseudomonas aeruginosa in health care facilities. Geneva: World Health Organization; 2017. Licence: CC BY—NC-SA 2013.2010 IGO.
19. Manchanda, V., Sanchaita. S., and Singh, N. (2010) Multidrug resistant Acinetobacter. J. Glob. Infect. Dis. 2, 291-304.
20. Doi, Y., Murray, G. L., and Peleg, A. Y. (2015) Acinetobacter baumannii: Evolution of antimicrobial resistance-treatment options, Semin. Respir. Crit. Care Med. 36, 85-98.
21. Clark, N. M., Zhanel, G. G., and Lynch, J. P., 3rd. (2016) Emergence of antimicrobial resistance among Acinetobacter species: A global threat, Curr. Opin. Crit. Care 22, 491-499.
22. Gordon, N. C., and Wareham, D. W. (2010) Multidrug-resistant Acinetobacter baumannii: Mechanisms of virulence and resistance, Int. J. Antimicrob. Agents 35, 219-226.
23. Moubareck, C. A., and Halat, D. H. (2020) Insights into Acinetobacter baumannii: A review of microbiological, virulence, and resistance traits in a threatening nosocomial pathogen, Antibiotics (Basel) 9, 119.
24. Alotaibi, T., Abuhaimed, A., Alshahrani, M., Albdelhady, A., Almubarak, Y., and Almasari, O. (2021) Prevalence of multidrug-resistant Acinetobacter baumannii in a critical care setting: A tertiary teaching hospital experience. SAGE Open Med 9, 20503121211001144.
25. Alrahmany, D., Omar, A. F., Alreesi, A., Harb, G., and Ghazi, I. M. (2022) Acinetobacter baumannii infection-related mortality in hospitalized patients: Risk factors and potential targets for clinical and antimicrobial stewardship interventions, Antibiotics (Basel) 11, 1086.
26. CDC. Antibiotic resistance threats in the United States. Atlanta, GA: U.S. Department of Health and Human Services, CDC; 2019.
27. Allison, D., Delancey, E., Ramey, H., Williams, C., Alsharif, Z. A., Al-Khattabi, H., Ontko, A., Gilmore, D., and Alam. M. A. (2017) Synthesis and antimicrobial studies of novel derivatives of 4-(4-formyl-3-phenyl-1H-pyrazol-1-yl)benzoic acid as potent anti-Acinetobacter baumannii agents, Bioorg. Med. Chem. Lett. 27, 387-392.
28. Alkhaibari, I., Kc, H. R., Angappulige, D. H., Gilmore, D., and Alam, M. A. (2022) Novel pyrazoles as potent growth inhibitors of staphylococci, enterococci and Acinetobacter baumannii bacteria, Future Med. Chem. 14, 233-244.
29. Rubin, H., Selwood, T., Yano, T., Weaver, D. G., Loughran, H. M., Costanzo, M. J., Scott, R. W., Wrobel, J. E., Freeman, K. B., and Reitz, A. B. (2015) Acinetobacter baumannii OxPhos inhibitors as selective anti-infective agents, Bioorg. Med. Chem. Lett. 25, 378-383.
30. Kwon, J., Mistry, T., Ren, J., Johnson. M. E., and Mehboob, S. (2018) A novel series of enoyl reductase inhibitors targeting the ESKAPE pathogens, Staphylococcus aureus and Acinetobacter baumannii, Bioorg. Med. Chem. 26, 65-76.
31. Soojhawon, I., Pattabiraman, N., Tsang, A., Roth, A. L., Kang, E., and Noble, S. M. (2017) Discovery of novel inhibitors of multidrug-resistant Acinetobacter baumannii, Bioorg. Med. Chem. 25, 5477-5482.
32. Steenackers, H. P., Parijs, I., Dubey, A., Foster, K. R., and Vanderleyden, J. (2016) Experimental evolution in biofilm populations, FEMS Microbiol. Rev. 40, 373-397.
33. Motyl, M.; Dorso, K.; Barrett, J.; Giacobbe, R. Curr. Protoc. Pharmacol. 2006, Chapter 13 (Unit 13A):13.
34. Shrestha, S. K.: Fosso, M. Y.; Green, K. D.; Gameau-Tsodikova, S. Amphiphilic tobramycin analogues as antibacterial and antifungal agents. Antimicrob. Agents Chemother. 2015, 59 (8), 4861-4869. DOI: 10.1128/AAC.00229-15 From NLM Medline.
35. Evans, B. C.; Nelson, C. E.; Yu, S. S.; Beavers, K. R.; Kim, A. J.; Li, H.; Nelson, H. M.; Giorgio, T. D.; Duvall, C. L. Ex vivo red blood cell hemolysis assay for the evaluation of pH-responsive endosomolvtic agents for cytosolic delivery of biomacromolecular drugs. J. Vis. Exp. 2013, (73), e50166. DOI: 10.3791/50166 From NLM Medline.
36. Green, K. D; Chandrika, N. T.; Vu, L. Y.; Pang, A. H.; Tsodikov, O. V.; Gameau-Tsodikova, S. Aromatic hydrazides: A potential solution for Acinetobacter baunannii infections, European Journal of Medicinal Chemistry, 2023, (249), 115165, doi.org/10.1016/j.ejmech.2023.115165.

1. A compound having the following formula or a pharmaceutically acceptable salt thereof: 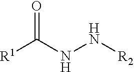
wherein,
R₁ is C₁₋₄ alkyl, C₁₋₄ alkene, 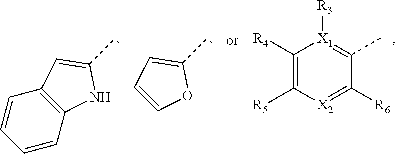
wherein
R₃, R₄, R₅, and R₆ are independently hydrogen or halogen, or R₄ and R₅, taken together with the atoms to which they are bound, form a 6-membered ring; and
X₁ and X₂ are independently C or N; and
R₂ is 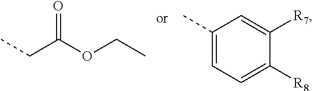
wherein
R₇ and R₈ are independently hydrogen, halogen, or C₁₋₄ alkoxy.

2. The compound of claim 1, having the following formula or a pharmaceutically acceptable salt thereof:

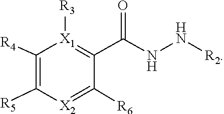

3. The compound of claim 2, wherein R₂ is

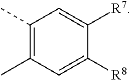

4. The compound of claim 2, wherein R₂ is

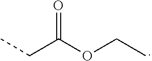

5. The compound of claim 1, having the following formula or a pharmaceutically acceptable salt thereof:

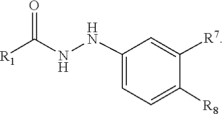

6. The compound of claim 5, wherein R₁ is

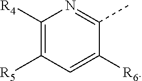

7. The compound of claim 5, wherein R₁ is

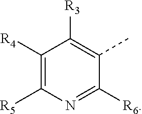

8. The compound of claim 5, wherein R₁ is

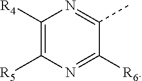

9. The compound of claim 5, wherein R₁ is

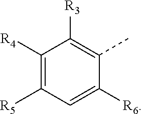

10. The compound of claim 5, wherein R₁ is

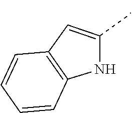

11. The compound of claim 5, wherein R₁ is

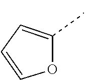

12. The compound of claim 5, wherein R₁ is C₁₋₄ alkyl or C₁₋₄ alkene.

13. The compound of claim 1, having the following formula or a pharmaceutically acceptable salt thereof.

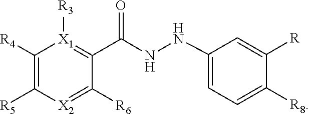

14. The compound of claim 1, one of the following formulae or a pharmaceutical acceptable salt thereof.

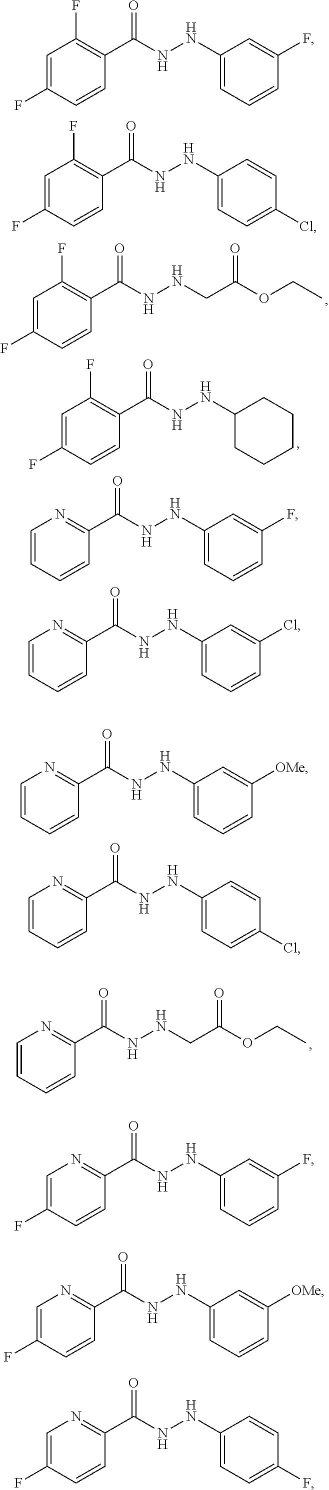

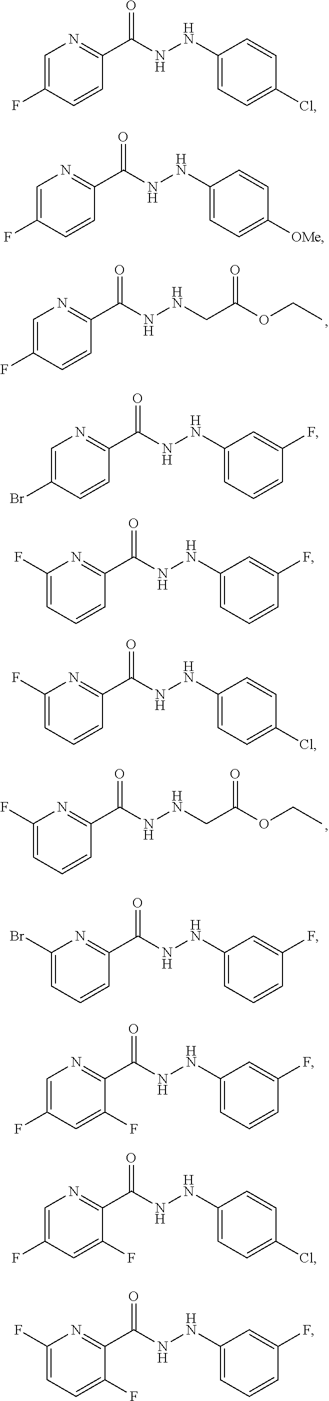

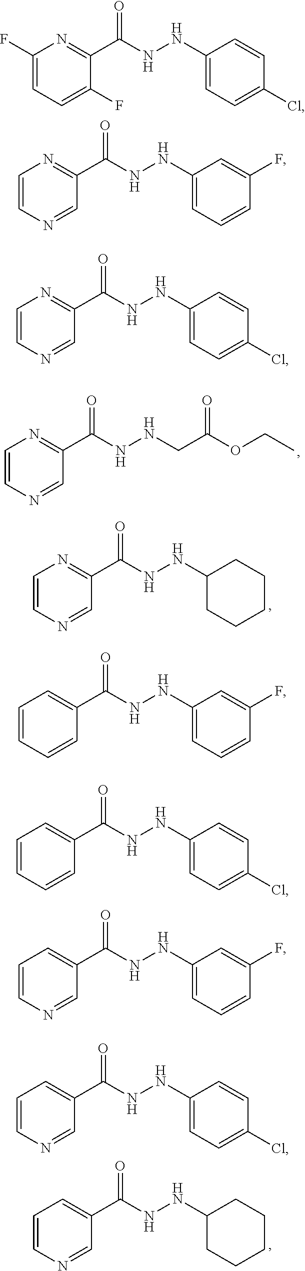

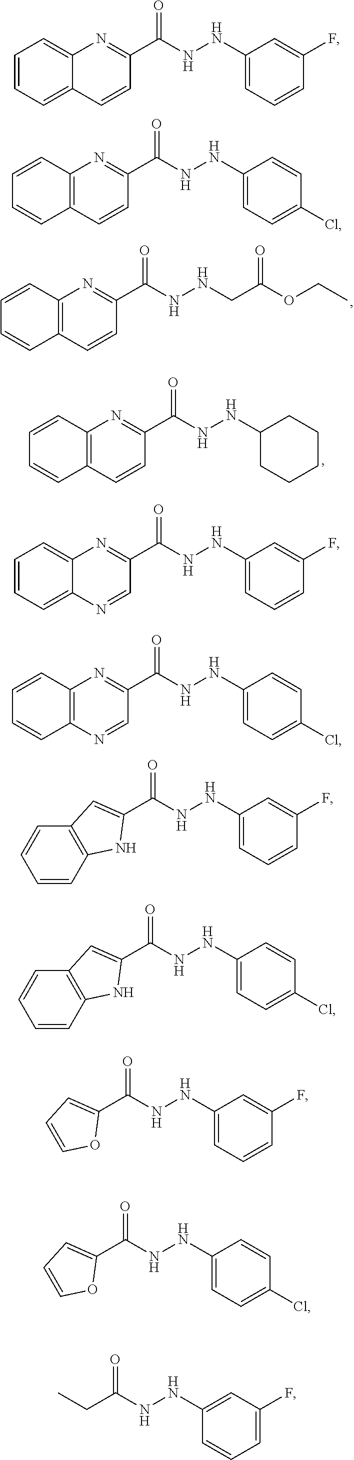

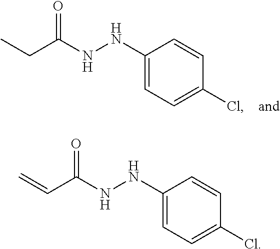

15. A pharmaceutical composition comprising the compound of claim 1, and a pharmaceutically-acceptable carrier.

16. A method of controlling Acinetobacter baumannii, comprising: contacting the microbe with an effective amount of the compound of claim 1.

17. (canceled)

18. A method of treating an Acinetobacter baumannii infection, comprising: administering to a subject in need thereof an effective amount of the compound of claim 1.

19. (canceled)

20. The method of claim 18, wherein the compound is administered prophylactically or therapeutically.
